(19) United States
(12) Patent Application Publication
(10) Pub. No.: US 2022/0251138 A1
(43) Pub. Date: Aug. 11, 2022

(54) PROCESSES FOR OBTAINING A HIGHLY CONCENTRATED ANTIBODY SOLUTION

(71) Applicant: ICHNOS SCIENCES SA, La Chaux-de-Fonds (CH)

(72) Inventors: Roberto GIOVANNINI, La Chaux-de-Fonds (CH); Anaïs DURET, La Chaux-de-Fonds (CH); Lionel DUARTE, La Chaux-de-Fonds (CH); Laure CAHUZAC, La Chaux-de-Fonds (CH); Thomas BERENGER, La Chaux-de-Fonds (CH); Sachin DUBEY, La Chaux-de-Fonds (CH); Benoit STROBBE, La Chaux-de-Fonds (CH)

(21) Appl. No.: 17/628,190

(22) PCT Filed: Jan. 22, 2020
(86) PCT No.: PCT/EP2020/051527

§ 371 Date: Jan. 18, 2022

(30) Foreign Application Priority Data
Jul. 19, 2019 (EP) PCT/EP2019/069522

(51) Int. Cl. C07K 1/36; C07K 16/06; C07K 1/22; C07K 1/18; C07K 1/34; A61K 39/395; A61K 47/22; A61K 47/18; A61K 47/26; B01D 15/38; B01D 15/36; B01D 61/14

(52) U.S. Cl. CPC C07K 1/36; B01D 2315/16; C07K 1/22; C07K 1/18; C07K 1/34; A61K 39/39591; A61K 47/22; A61K 47/183; A61K 47/26; B01D 15/3809; B01D 15/362; B01D 61/145; B01D 61/142; B01D 15/363; B01D 2315/10; B01D 2311/2626; B01D 2311/2623; C07K 16/065

(57) ABSTRACT

The present invention relates to processes for obtaining a highly concentrated antibody solution. In particular, to processes for obtaining a highly concentrated therapeutic antibody solution that may be used for highly concentrated therapeutic antibody formulations, e.g. suitable for subcutaneous administration.

### TECHNICAL FIELD

The present invention relates to processes for obtaining a highly concentrated antibody solution. In particular, to processes for obtaining a highly concentrated therapeutic antibody solution that may be used for highly concentrated therapeutic antibody formulations, e.g. suitable for subcutaneous administration.

### BACKGROUND

The increasing use of proteins, such as antibodies, as pharmaceuticals for clinical applications, has made the development of high efficient purification methods crucial for their manufacturing. Typically, therapeutic proteins, such as antibodies are produced by cells, using for instance mammalian or bacterial cells engineered so as to express the protein of interest and cultured under controlled conditions that aid their growth and the expression of the protein of interest. The result of the cell culture is a complex mix comprising, besides the host cells expressing the protein of interest and the protein of interest itself, cell debris, colloidal particles, such as DNA, RNA and host cell proteins (HCP), and other (bio)molecules secreted by the cultured cells.

Separating the protein of interest from this mix is not a simple operation especially considering the optimized upstream processes that lead to higher titers of the biomolecule of interest at the end of the cell culture as well as to increased level of contaminant species as the cells are stresses due to the optimized upstream process. As consequence, the downstream purification process normally requires multiple steps, such as chromatography, filtration, viral removal, and it might also include steps to concentrate the protein to the desired concentration level. The selection of an effective downstream purification sequence is crucial the development and manufacturing of highly purified and safe therapeutics, especially at large scales.

For therapeutic antibodies, the downstream process is in many cases designed so as to obtain antibody concentrations in the range of about 1-60 mg/mL (WO2016/063299, WO2014/207763), which are suitable when, after formulation, lower concentrations of the antibody in the drug product are desired. Nevertheless, in certain situations higher concentrations of an antibody in the drug produce are necessary, for instance, when therapeutic antibody preparations are made for subcutaneous delivery, highly concentrated antibody formulations may be necessary to avoid multiple injections and still obtaining the expected therapeutic effect. The needed antibody concentration in these cases may be about 100 mg/mL or above, meaning that the purification process should be implemented so as to obtain higher concentrated solutions. Despite this necessity, implementing a downstream purification process that allows to reach high antibody concentrations still remains a challenge.

### SUMMARY

The present invention relates to a process for obtaining a highly concentrated antibody solution comprising the steps of subjecting a clarified cell harvest to an affinity chromatography step, and subjecting the obtained eluate to at least two ion exchange chromatography steps and to at least three UF/DF steps.

In particular the highly concentrated antibody solution obtained by the process of the present invention has an antibody concentration equal to or greater than about 120 g/L.

In one embodiment of the current disclosure, the at least three UF/DF steps are performed with a tangential flow filtration cassette and comprise a first UF/DF performed after the first of said at least two ion exchange chromatography, a second UF/DF performed after the second of said at least two ion exchange chromatography, and a third UF/DF performed after the second UF/DF, and wherein said highly concentrated antibody solution has an antibody concentration equal to or greater than about 150 g/L.

In a more particular embodiment of the present invention, the third UF/DF comprises the steps of:
(a) equilibration of the cassette by an equilibration buffer;
(b) loading of the cassette with an antibody solution with antibody concentration comprised between about 50 g/L and about 90 g/L;
(c) first ultrafiltration to concentrate the antibody to a concentration comprised between about 80 g/L and about 120 g/L;
(d) diafiltration using a diafiltration buffer;
(e) second ultrafiltration to concentrate the antibody to a concentration comprised between about 200 g/L and about 300 g/L;
(f) flushing of the cassette with a flushing buffer;
(g) obtaining an highly concentrated antibody solution with antibody concentration comprised between about 150 g/L and about 200 g/L.

More in particular, the antibody solution loaded onto the third UFDF cassette has an antibody concentration of about 70 g/L and/or the first ultrafiltration concentrates the antibody to a concentration of about 100 g/L and/or the second ultrafiltration concentrates the antibody to a concentration of about 260 mg/mL and/or the obtained highly concentrated antibody solution has an antibody concentration of about 170 g/L.

In certain specific embodiment, the third UF/DF is performed using an equilibration buffer comprising histidine-HCl at a concentration of about 5 mM and having pH about 6, a diafiltration buffer comprising histidine-HCl at a concentration of about 25 mM and arginine-HCl at a concentration of about 150 mM and having pH of about 6 and flushing buffer comprising histidine-HCl at a concentration of about 25 mM and arginine-HCl at a concentration of about 150 mM and having pH of about 6.

In one aspect, the affinity chromatography is protein A affinity chromatography.

In another aspect, the at least two steps of ion exchange chromatography steps comprise a first step of cation exchange chromatography and a second step of anion exchange chromatography.

In a further aspect, the antibody according to the present invention is an antibody or fragment thereof.

The present invention also relates to a stable pharmaceutical formulation obtained by adding excipients to the highly concentrated antibody solution obtained by the process disclosed herein.

More in particular, the present disclosure relates to a stable pharmaceutical formulation comprising an a antibody or fragment thereof present within said pharmaceutical formulation at a concentration of about 150 g/L, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v).

Disclosed herein is also a process of production of a bulk drug substance or a drug product comprising the steps of:
(a) Protein A chromatography of a clarified cell harvest comprising an antibody;
(b) Viral inactivation of the resulting protein A eluate;
(c) Neutralization of the protein A eluate to pH 5.2, followed by 0.2 μm filtration;
(d) Cation exchange chromatography of the neutralized protein A eluate, followed by 0.2 μm filtration;
(e) First UF/DF of the cation exchange chromatography eluate, followed by 0.2 μm filtration;
(f) Anion exchange chromatography in flow through mode performed by membrane adsorption, followed by 0.2 am filtration;
(g) Viral nanofiltration;
(h) Second UF/DF of the nanofiltrated solution, followed by 0.2 um filtration;
(i) Third UF/DF of the antibody solution obtained by the second UF/DF according to the processes for obtaining a highly concentrated antibody solution as disclosed herein, followed by 0.2 um filtration;
(j) Obtaining a stable pharmaceutical formulation by adding excipients to the highly concentrated antibody solution obtained by the third UF/DF, followed by 0.2 um filtration.

More specifically it is disclosed a process of production of a bulk drug substance or a drug product wherein the third UF/DF is performed using an equilibration buffer comprising histidine-HCl at a concentration of about 5 mM and having pH about 6, a diafiltration buffer comprising histidine-HCl at a concentration of about 25 mM and arginine-HCl at a concentration of about 150 mM and having pH of about 6 and flushing buffer comprising histidine-HCl at a concentration of about 25 mM and arginine-HCl at a concentration of about 150 mM and having pH of about 6, and wherein the stable pharmaceutical formulation comprises an a antibody or fragment thereof present within said pharmaceutical formulation at a concentration of about 150 g/L, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v).

As used herein, the following terms have the following meanings: “a”, “an”, and “the” as used herein refers to both singular and plural unless the context clearly dictates otherwise.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of cell and tissue culture, molecular biology, and protein and oligo- or polynucleotide chemistry, laboratory procedures and techniques of analytical chemistry, synthetic organic chemistry, and medicinal and pharmaceutical chemistry described herein are those well-known and commonly used in the art.

The present invention relates to a process for obtaining a highly concentrated antibody solution.

According to the present invention the term “highly concentrated antibody solution” or “high concentration antibody solution” refers to a solution containing an antibody at a concentration equal to or greater than about 50 g/L; for instance, to a solution containing an antibody at a concentration equal to or greater than about 60 g/L, or at concentration equal to or greater than about 70 g/L, or at concentration equal to or greater than about 80 g/L, or at concentration equal to or greater than about 100 g/L, or at concentration equal to or greater than about 120 g/L, or at concentration equal to or greater than about 150 g/L, or at concentration equal to or greater than about 170 g/L, or at concentration equal to or greater than about 200 g/L, or at concentration equal to or greater than about 250 g/L. More in particular the highly concentrated antibody solution of the present invention comprises an antibody at a concentration selected from the group comprising: about 50 g/L, about 60 g/L, about 70 g/L, about 80 g/L, about 90 g/L, about 100 g/L, about 110 g/L, about 120 g/L, about 130 g/L, about 140 g/L, about 150 g/L, about 160 g/L, about 170 g/L, about 180 g/L, about 190 g/L, about 200 g/L, about 210 g/L, about 220 g/L, about 230 g/L, about 240 g/L, about 250 g/L, about 260 g/L, about 270 g/L, about 280 g/L, about 290 g/L, about 300 g/L, about 310 g/L, about 320 g/L, about 330 g/L, about 340 g/L, about 350 g/L, about 360 g/L, about 370 g/L, about 380 g/L, about 390 g/L, about 400 g/L. In certain preferred embodiments, the highly concentrated antibody solution obtained by the process according to the present invention has an antibody concentration selected from the group comprising about 120 g/L, about 150 g/L, about 160 g/L, about 170 g/L, about 180 g/L, about 200 g/L, in a particularly preferred embodiment, the highly concentrated antibody solution obtained by the process according to the present invention has an antibody concentration of about 170 g/L. The present invention also discloses highly concentrated antibody solution with antibody concentration at any intermediate value of the above cited values.

In particular the present invention relates to a process for obtaining a highly concentrated antibody solution comprising the steps of subjecting a clarified cell harvest to an affinity chromatography step, and subjecting the obtained eluate to at least two ion exchange chromatography steps and to at least three UF/DF steps.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The term “heavy chain variable framework region” as referred herein may comprise one or more (e.g., one, two, three and/or four) heavy chain framework region sequences (e.g., framework 1 (FW1), framework 2 (FW2), framework 3 (FW3) and/or framework 4 (FW4)). Preferably the heavy chain variable region framework comprises FW1, FW2 and/or FW3, more preferably FW1, FW2 and FW3. The term “light chain variable framework region” as referred herein may comprise one or more (e.g., one, two, three and/or four) light chain framework region sequences (e.g., framework 1 (FW1), framework 2 (FW2), framework 3 (FW3) and/or framework 4 (FW4)). Preferably the light chain variable region framework comprises FW1, FW2 and/or FW3, more preferably FW1, FW2 and FW3. The variable regions of the heavy and light chains contain a binding domain that interacts with an antigen. The constant regions of the antibodies may mediate the binding of the immunoglobulin to host tissues or factors, including various cells of the immune system (e.g., effector cells) and the First component (C1q) of the classical complement system.

Antibodies are grouped into classes, also referred to as isotypes, as determined genetically by the constant region. Human constant light chains are classified as kappa (CK) and lambda (CX) light chains. Heavy chains are classified as mu (μ), delta (δ), gamma (γ), alpha (a), or epsilon (ε), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgG1 (IGHG1), IgG2 (IGHG2), IgG3 (IGHG3), IgG4 (IGHG4), IgA1 (IGHA1), IgA2 (IGHA2), IgM (IGHM), IgD (IGHD), and IgE (IGHE). The so-called human immunoglobulin pseudo-gamma IGHGP gene represents an additional human immunoglobulin heavy constant region gene which has been sequenced but does not encode a protein due to an altered switch region (Bensmana M et al., (1988) Nucleic Acids Res. 16(7): 3108). In spite of having an altered switch region, the human immunoglobulin pseudo-gamma IGHGP gene has open reading frames for all heavy constant domains (CH1-CH3) and hinge. All open reading frames for its heavy constant domains encode protein domains which align well with all human immunoglobulin constant domains with the predicted structural features. This additional pseudo-gamma isotype is referred herein as IgGP or IGHGP. Other pseudo immunoglobulin genes have been reported such as the human immunoglobulin heavy constant domain epsilon PI and P2 pseudo-genes (IGHEP1 and IGHEP2). The IgG class is the most commonly used for therapeutic purposes. In humans this class comprises subclasses IgG1, IgG2, IgG3 and IgG4. In mice this class comprises subclasses IgG1, IgG2a, IgG2b, IgG2c and IgG3.

Antibody fragments include, but are not limited to, (i) the Fab fragment consisting of VL, VH, CL and CHI domains, including Fab′ and Fab′-SH, (ii) the Fd fragment consisting of the VH and CHI domains, (iii) the Fv fragment consisting of the VL and VH domains of a single antibody; (iv) the dAb fragment (Ward E S et al., (1989) Nature, 341: 544-546) which consists of a single variable, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al, (1988) Science 242: 423-426; Huston J S et al, (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al, (1993) Proc. Natl. Acad. Sci. USA, 90: 6444-48) and (ix) scFv genetically fused to the same or a different antibody (Coloma M J & Morrison S L (1997) Nature Biotechnology, 15(2): 159-163).

An antibody can be produced by introducing genetic material encoding said biomolecule of interest in host cells and culture said host cells. The term “host cells” refers to all the cells in which the biomolecule of interest, such as an antibody or antibody fragment thereof, codified by the artificially introduced genetic material is expressed, including those cells in which the foreign nucleic acid is directly introduced and their progeny. In the host cells it can be introduced an expression vectors (constructs), such as plasmids and the like, encoding the biomolecule of interest e.g., via transformation, transfection, infection, or injection. Such expression vectors normally contain the necessary elements for the transcription and translation of the sequence encoding the biomolecule of interest. Methods which are well known to and practiced by those skilled in the art can be used to construct expression vectors containing sequences encoding the protein of interest, as well as the appropriate transcriptional and translational control elements. These methods include in vitro recombinant DNA techniques, synthetic techniques, and in vivo genetic recombination. Cell lines suitable as host cells include and are not limited to bacteria, mammalian, insect, plant and yeast cells. Cell lines often used for the expression and production of therapeutic antibodies include mammalian cells lines such as Chinese hamster ovary (CHO) cells, NSO mouse myeloma cells, human cervical carcinoma (HeLa) cells and human embryonic kidney (HEK) cells.

The terms “cell culture” and “culture” as used herein are interchangeable and refer to the growth and/or propagation and/or maintenance of cells in controlled artificial conditions, and they indicate a cell culture which comprises a cell culture medium and cell culture material comprising cells, cell debris, for instance generated upon cell death, colloidal particles, such as DNA, RNA and host cell proteins (HCP), and (bio)molecules secreted by the cultured cells, such as the biomolecule of interest. The cells of a cell culture can be cultured in suspension or attached to a solid substrate, in containers comprising a cell culture medium. For example a cell culture can be grown in tubes, spin tubes, flasks, bags, roller bottles, bioreactors.

When the production of the biomolecule of interest has a commercial purpose, often the host cells are cultured in bioreactors, under conditions that aid their growth and the expression of said biomolecule of interest. The term “bioreactor,” as used herein, refers to any manufactured or engineered device or system that supports a biologically active environment. Optimal culturing conditions are obtained by the control and adjustment of several parameters including: the formulation of the cell culture medium, the bioreactor operating parameters, the nutrient supply modality and the culturing time period. The formulation of the culturing medium has to be optimized to favorite cell vitality and reproduction; examples of constituents of the cell culture medium include but are not limited to essential amino acids, salts, glucose, growth factors and antibiotics. Important bioreactor operating parameters include: initial cell seeding density, temperature, pH, agitation speed, oxygenation and carbon dioxide levels. Nutrients can be supplied in different ways: in the batch mode culture all the necessary nutrients are present in the initial base medium and are used till exhausted while wastes accumulate; in the fed-batch culture additional feed medium is supplied to prevent nutrient depletion and prolong the culture; differently, in the perfusion modality, cells in culture are continuously supplemented with fresh medium containing nutrients that flows in the bioreactor removing cell wastes. The culturing period is important as it needs to be long enough to let the cells produce a consistent amount of product but it cannot be too long to impair their viability. A non-limiting example of the duration of a culturing period is between about 10 and about 18 days, specifically between about 11 days and 15 days, more specifically between about 12 days and 14 days. For instance the culturing period is a period selected from the group comprising about 10 day, about 11 days, about 12 days, about 13 days, about 14 days, about 15 days, about 16 days, about 17 days, about 18 days, preferably for 14 days. Preferred culturing periods are selected from the group comprising about 11 days, about 12 days, about 13 days, about 14 days, about 15 days. Most preferred culturing periods are selected from the group comprising about 12 days, about 13 days, about 14 days. Commonly used bioreactors are typically cylindrical, ranging in size from liters to cubic meters, and may be made of stainless steel or plastic. It is contemplated that the total volume of a bioreactor may be any volume ranging from 100 mL to up to 20000 Liters or more, depending on a particular process. Non limiting examples of bioreactor volumes include about 100 mL, about 200 mL, about 500 mL, about 800 mL, about 1 L, about 5 L, about 10 L, about 20 L, about 30 L, about 40 L, about 50 L, about 60 L, about 70 L, about 80 L, about 90 L, about 100 L, about 200 L, about 300 L, about 400 L, about 500 L, about 600 L, about 700 L, about 800 L, about 900 L, about 1000 L, about 2000 L, about 3000 L, about 4000 L, about 5000 L, about 6000 L, about 7000 L, about 8000 L, about 9000 L, about 10000 L, about 15000 L, about 20000 L. In a preferred embodiment of the present invention the bioreactor has a volume comprised between about 1000 L and 15000 L, more preferably comprised between about 1000 L and 10000 L, even more preferably the volume is about 5000 L.

The terms “cell culture medium,” and “culture medium” and “medium” are used interchangeably herein and they refer to a nutrient solution used for growing cells, such as animal cells, e.g., mammalian cells. Such a nutrient solution generally includes various factors necessary for cell attachment, growth, and maintenance of the cellular environment. For example, a typical nutrient solution may include a basal media formulation, various supplements depending on the cell type and, occasionally, antibiotics. During cell culture the cell culture medium may also contain cell culture material such as cell waste products, host cell proteins (HCP) and material from lysed cells. The composition of the culture medium may vary in time during the course of the culturing of cells.

The terms “clarify”, “clarification”, “clarification step”, “clarification process” as used herein are interchangeable and generally they refer to one or more steps that aid the removal of a part of the cell culture material from the cell culture, such as removal of cells, cell debris and colloidal particles, to obtained clarified cell culture, also called clarified cell culture fluid (CCCF), comprising the biomolecule of interest.

The term “clarified cell harvest” refers to a material produced by first harvesting the host cell culture and then subjecting the harvest to a process of clarification.

The clarified cell harvest may be loaded onto a chromatography column for further purification.

The term “chromatography” refers to the operation of separating compounds of a mixture based on their capability to interact with a stationary phase of a chromatography, from which they can be retained or eluted. Non limiting examples of chromatographic techniques, including ion exchange, hydrophobic interaction, affinity, sizing or gel filtration, and reversed-phase chromatography, carried out at atmospheric pressure or at high pressure using systems such as FPLC and HPLC

The process of “affinity chromatography” involves the use of an affinity reagent as ligands which are cross-linked to the stationary phase and that have binding affinity to specific molecules or a class of molecules. Ligands can be bio-molecules, like protein ligands or can be synthetic molecules. Both types of ligand tend to have good specificity. The most commonly used protein ligand in production is the affinity reagent Protein A. In affinity chromatography when the solution (for example a crude cell supernatant containing a protein of interest) is loaded onto to the column the target protein is usually adsorbed while allowing contaminants (other proteins, lipids, carbohydrates, DNA, pigments, etc.) to pass through the column. The adsorbent itself is normally packed in a chromatography column; though the adsorption stage can be performed by using the adsorbent as a stirred slurry in batch binding mode. The next stage after adsorption is the wash stage, in which the adsorbent is washed to remove residual contaminants. The bound protein is then eluted in a semi-pure or pure form. Elution is normally achieved by changing the buffer or salt composition so that the protein can no longer interact with the immobilized ligand and is released. Affinity chromatography can be performed in a fixed bed or a fluidized bed.

Ion exchange chromatography separates ions and polar molecules based on their affinity to the ion exchanger, example of ion exchange chromatography techniques are cation exchange chromatography and cation exchange chromatography. Cation-exchange chromatography (CEX) is used when the molecule of interest is positively charged. In this case, the stationary phase is negatively charged and positively charged molecules are loaded to be attracted to it. In the anion-exchange chromatography (AEX) the stationary phase is positively charged and negatively charged molecules are attracted to it. Ion exchange chromatography can be performed on chromatography columns (resins) or using membrane absorbers (MA). In one aspect of the present invention the at least two steps of ion exchange chromatography steps comprise a first step of cation exchange chromatography and a second step of anion exchange chromatography. In particular cation exchange chromatography (CEX) is carried out in bind-eluate mode wherein the antibody is eluted with an elution buffer selected from the group comprising Tris, Citrate, Acetate, Histidine and Phosphate and anion exchange chromatography is carried out by membrane absorption (MA) in flow-through mode using a buffer selected from the group comprising Tris, Citrate, Acetate, Histidine and Phosphate.

Ultrafiltration (UF) and diafiltration (DF) refer to the steps that allow protein concentration and buffer exchange, more specifically UF/DF concentrates and resuspends the product in a desired buffer. Normally the solution is contacted with the membrane under an applied pressure, which forces salts and molecules smaller than the membrane pores to pass through the membrane while the membranes retain the proteins. UF/DF can be performed in a tangential flow filtration (TFF) with cassettes or normal flow filtration (NFF). NFF could be carried out by dead-end filters or cartridge filters. TFF may consists in a filtration cassette inside which the flows between two membranes, the flow inside the cassette also generates pressure which applied to the flow perpendicularly, i.e. against the membranes walls, this pressure pushes solvent through the membrane toward the permeate line, and molecules smaller than the membrane cut-off with it, while bigger molecules are kept in recirculation back to the feed/retentate or through the drain. In preferred embodiments of this application the UF/DF step(s) is performed by TFF cassettes. In certain embodiments the UF/DF cassette membrane has a nominal molecular weight limit (NMWL) selected from the group comprising about 3 kDa, about 5 kDa, about 10 kDa, about 30 kDa, about 50 kDa. In a preferred embodiment the NMWL is 30 kDa. The present invention also includes NMWL values at any intermediate value of the above said value.

The process according to the current invention allows to obtain a highly concentrated antibody solution with an antibody concentration equal to or greater than about 50 g/L, preferably equal to or greater than about 60 g/I, more preferably equal to or greater than about 100 g/L, even more preferably equal to or greater than about 120 g/L, particularly preferably equal to or greater than about 150 g/L, specifically equal to or greater than 170 g/L. In certain preferred embodiments the antibody solution has an antibody concentration comprised between about 60 g/L and about 400 g/L, in particular the antibody concentration is comprised between about 100 g/L and about 300 g/L, or comprised between about 120 g/L and about 350 g/L, or comprised between about 120 g/L and about 200 g/L, or comprised between about 150 g/L and about 200 g/L, or comprised between about 160 g/L and about 180 g/L, or comprised between about 162 g/L and about 179 g/L. According to an aspect of the present invention, the antibody solution obtained by the process disclosed herein has an antibody concentration selected from the group comprising about 60 g/L, about 80 g/L, about 100 g/L, about 120 g/L, about 150 g/L, about 160 g/L, about 170 g/L, about 180 g/L, about 200 g/L, about 220 g/L, about 250 g/L, about 280 g/L, about 300 g/L, about 320 g/L, about 350 g/L, about 380 g/L, about 400 g/L. In a more preferred embodiment the antibody solution obtained by the process of the present invention has an antibody concentration of about 150 g/L, or of about 160 g/L, or of about 170 g/L or of about 180 g/L, most preferably of about 170 g/L. The present invention also includes antibody concentrations values at any intermediate value of the above said value.

According to the process of the present invention the antibody solution subjected to the last of the at least three UFDF steps has an antibody concentration comprised between about 50 g/L and 100 g/L, specifically the between about 60 g/ and about 80 g/L, preferably between about 65 g/L and 75 g/L, for instance the antibody solution subjected to the last of the at least three UFDF steps has an antibody concentration selected from the group comprising about 50 g/L, about 55 g/L, about 60 g/L, about 65 g/L, about 70 g/L, about 75 g/L, about 80 g/L, about 85 g/L, about 90 g/L, about 95 g/L, about 100 g/L; in a particular embodiment of the present invention, the antibody solution subjected to the last of the at least three UFDF steps has an antibody concentration of about 70 g/L.

In one aspect, the process according to the present invention comprises at least three UF/DF steps which are performed with a tangential flow filtration cassette, and at least two ion exchange chromatography steps. In particular the at least three UF/DF steps comprise a first UF/DF performed after the first of the at least two ion exchange chromatography, a second UF/DF performed after the second of the at least two ion exchange chromatography, and a third UF/DF performed after the second UF/DF.

According to the process of the present invention the antibody solution subjected to the third UFDF has an antibody concentration comprised between about 50 g/L and about 100 g/L, specifically between about 50 g/L and about 90 g/L, more specifically the between about 60 g/and about 80 g/L, preferably between about 65 g/L and 75 g/L; for instance the antibody solution subjected to the third UFDF has an antibody concentration selected from the group comprising about 50 g/L, about 55 g/L, about 60 g/L, about 65 g/L, about 70 g/L, about 75 g/L, about 80 g/L, about 85 g/L, about 90 g/L, about 95 g/L, about 100 g/L; in a particular embodiment of the present invention, the antibody solution subjected to the third UFDF has an antibody concentration of about 70 g/L.

In a particular aspect of the current disclosure, the antibody solution obtained after the third UF/DF step of the process of the present invention has an antibody concentration equal o or greater than about 60 g/L, preferably equal to or greater than about 100 g/L, more preferably equal to or greater than about 120 g/L, even more preferably equal to or greater than about 150 g/L, specifically equal to or greater than 170 g/L. In certain preferred embodiments the antibody solution has an antibody concentration comprised between about 60 g/L and about 400 g/L, in particular the antibody concentration is comprised between about 100 g/L and about 300 g/L, or comprised between about 120 g/L and about 250 g/L, or comprised between about 120 g/L and about 200 g/L, or comprised between about 150 g/L and about 200 g/L, or comprised between about 160 g/L and about 180 g/L, or comprised between about 162 g/L and about 179 g/L. According to an aspect of the present invention, the antibody solution obtained after the third UF/DF step of the process disclosed herein has an antibody concentration selected from the group comprising about 60 g/L, about 80 g/L, about 100 g/L, about 120 g/L, about 150 g/L, about 160 g/L, about 170 g/L, about 180 g/L, about 200 g/L, about 220 g/L, about 250 g/L, about 280 g/L, about 300 g/L, about 320 g/L, about 350 g/L, about 380 g/L, about 400 g/L. In a more preferred embodiment the antibody solution obtained after the third UF/DF step of the process of the present invention has an antibody concentration of about 150 g/L, or of about 160 g/L, or of about 170 g/L or of about 180 g/L, most preferably of about 170 g/L. The present invention also includes antibody concentrations values at any intermediate value of the above said value.

According to one embodiment of the present invention, the third UFDF comprises the steps of (a) equilibration of the cassette by an equilibration buffer; (b) loading of the cassette with an antibody solution with antibody concentration comprised between about 50 g/L and about 100 g/L, preferably between about 60 g/L and about 80 g/L, more preferably between about 65 g/L and about 75 g/L, for instance the antibody concentration is selected from the group comprising about 50 g/L, about 55 g/L, about 60 g/L, about 65 g/L, about 70 g/L, about 75 g/L, about 80 g/L, about 85 g/L, about 90 g/L, about 95 g/L, about 100 g/L, in a particular embodiment the antibody solution has an antibody concentration of about 70 g/L; (c) first ultrafiltration to concentrate the antibody to a concentration between about 80 g/L and about 120 g/L, preferably between about 90 g/L and about 110 g/L, for instance the antibody concentration after the first ultrafiltration is selected from the group comprising about 80 g/L, about 85 g/L, about 90 g/L, about 95 g/L, about 100 g/L, about 105 g/L, about 110 g/L, about 115 g/L, about 120 g/L, in a particular embodiment the antibody concentration after the first ultrafiltration of about 100 g/L; (d) diafiltration using a diafiltration buffer; (e) second ultrafiltration to concentrate the antibody to a concentration between about 200 g/L and about 300 g/L, for instance to a concentration selected from the group comprising about 200 g/L, about 220 g/L, about 240 g/L, of about 260 mg/mL, about 280 g/L, about 300 g/L, preferably to a concentration of about 260 g/L; (f) flushing of the cassette with a flushing buffer; (g) obtaining an highly concentrated antibody solution with antibody concentration comprised between about 60 g/L and about 400 g/L, preferably between about 100 g/L and about 300 g/L, more preferably comprised between about 120 g/L and about 250 g/L, even more preferably comprised between about 120 g/L and about 200 g/L, or comprised between about 150 g/L and about 200 g/L, particularly preferably comprised between about 160 g/L and about 180 g/L, for example comprised between about 162 g/L and about 179 g/L, for instance the obtained antibody solution has an antibody concentration selected from the group comprising about 60 g/L, about 80 g/L, about 100 g/L, about 120 g/L, about 150 g/L, about 160 g/L, about 170 g/L, about 180 g/L, about 200 g/L, about 220 g/L, about 250 g/L, about 280 g/L, about 300 g/L, about 320 g/L, about 350 g/L, about 380 g/L, about 400 g/L, in a preferred embodiment the antibody solution obtained after the third UF/DF step of the process of the present invention has an antibody concentration of about 150 g/L, or of about 160 g/L, or of about 170 g/L or of about 180 g/L, most preferably of about 170 g/L. The present invention also discloses antibody concentration at any value of the above mentioned values.

In certain specific embodiments, the antibody solution loaded onto the third UFDF cassette has an antibody concentration of about 70 g/L and/or the first ultrafiltration concentrates the antibody to a concentration of about 100 g/L and/or the second ultrafiltration concentrates the antibody to a concentration of about 260 mg/mL and/or the obtained antibody solution has an antibody concentration of about 170 g/L.

In an even more specific embodiments, the loading of the third UFDF cassette is performed at room temperature using a volumetric loading factor equal to or less than about 40 L/m², preferably equal to or less than about 30 L/m², preferably of about 25 L/m²; the first ultrafiltration and the diafiltration are performed at a cross flow rate (CFR) comprised between about 70 LMH and 350 LMH, preferably between about 100 LMH and 325 LMH, more preferably at a cross flow rate of about 290 LMH, at a feed flow rate (FFR) comprised between about 80 LMH and about 400 LMH, preferably comprised between about 110 LMH and about 350 LMH, more preferably at a FFR of about 315 LMH, with a feed pressure equal to or less than about 5 bars, preferably equal to or less than about 3 bars and a transmembrane pressure (TMP) comprised between about 0.3 bars and about 1.5 bars, preferably comprised between 0.6 bars and 1 bar, more preferably with a TMP of about 0.8 bars, and diafiltration is performed at a number of diafiltration volume (DV) equal to or greater than about 5, preferably equal to or greater than about 6, for instance for instance at 5 DVs, 6 DVs, 7 DVs or 8 Dvs; the second ultrafiltration is performed at a cross flow rate (CFR) between about 1 LMH and 350 LMH, preferably between about 7 LMH and between about 325 LMH, more preferably at a CFR of about 290 LMH, a feed flow rate (FFR) comprised between about 1 LMH and about 400 LMH, preferably comprised between about 7 LMH and about 350 LMH, more preferably at a FFR of about 315 LMH, with a feed pressure equal to or less than about 5 bars, preferably equal or less than about 3 bars and a TMP equal to or less than about 3 bars, preferably equal to or less than about 1.5 bars. The present invention also includes volumetric loading factor, cross flow rate, feed flow rate, feed pressure at any value between the above cited values.

The selection of the equilibration, diafiltration and flushing buffer is related to the desired antibody formulation. In a specific embodiment of the present invention, the equilibration buffer comprising L-histidine at a concentration comprised between about 1 and about 10 mM, preferably at a concentration of about 5 mM at a pH comprised between 5 and 7, preferably at a of pH about 6, a diafiltration buffer comprising L-histidine at a concentration comprised between about 10 mM and about 50 mM, preferably at a concentration of about 25 mM and L-arginine-HCl at a concentration comprised between about 100 mM and about 200 mM, preferably at a concentration of about 150 mM at a pH comprised between 5 and 7, preferably at a of, and a flushing buffer comprising L-histidine at a concentration comprised between about 10 mM and about 50 mM, preferably at a concentration of about 25 mM and L-arginine-HCl at a concentration comprised between about 100 mM and about 200 mM, preferably at a concentration of about 150 mM at a pH comprised between 5 and 7, preferably at a of. The present invention also includes buffers components with a concentration and pH at any value between the above cited concentrations.

In a particularly specific embodiment, the third UFDF is performed according to the following steps:

### Sanitation:

Pre-Use water for injection (WFI) flush
Sanitation with 0.5 NaOH-30 min recirculation
Pre-use WFI rinsing ≤0.1 mS/cm
Feed pressure: 3 bars
TMP target 0.8 bars (0.6 bars-≤1. bars)

### Equilibration:

Equilibration buffer: 5 mM L-Histidine pH 6.0
Feed pressure: 3 bars
TMP target 0.8 bars (0.6 bars-≤1. bars)

### Loading:

Volumetric loading factor: target 25 L/m2 (≤30 L/m2)
Concentration: ≥65 g/L-≤75 g/L

### First Ultrafiltration:

Cross flow rate: target 290 LMH (≥100 LMH-≤325 LMH)
Feed flow rate: target 315 LMH (≥110 LMH-≤350 LMH)
Feed pressure: ≤3 bars
TMP: target 0.8 bars (≥0.6 bars-≤1.0 bars)
Concentration: target 100 g/L (≥90 g/L-≤110 g/L)

### Diafiltration:

Cross flow rate: target 290 LMH (≥100 LMH-≤325 LMH)
Feed flow rate: target 315 LMH (≥110 LMH-≤350 LMH)
Feed pressure: ≤3 bars
TMP: target 0.8 bars (≥0.6 bars-≤1.0 bars)
Diafiltration buffer: 25 mM L-Histidine, 150 mM L-Arginine-HCl pH 6.0, performed at ≥6 DVs

### Second Ultrafiltration:

Cross flow rate: target 290 LMH (≥7 LMH≤325 LMH)
Feed flow rate: target 315 LMH (≥7 LMH-≤350 LMH)
Feed pressure: ≤3 bars
TMP: ≤1.5 bars
Concentration: ≤260 g/L

### Flushing:

Flushing buffer: 25 mM L-Histidine, 150 mM L-Arginine-HCl pH 6.0-≥3 Hold-Up Volume (HUV)
Concentration reached: ≥162 g/L-≤179 g/L

In another particular embodiment, the UFDF3 concentrated solution is formulated to obtain an antibody concentration between about 100 g/L and about 200 g/L, preferably between about 160 g/L and about 170 g/L, more preferably the formulated antibody concentration is about 150 g/L. In a more specific embodiment, the UFDF3 concentrated solution is formulated by the addition of one or more excipient(s), for instance by the addition of one or more stabilizing or tonicity agent(s), such as Polysorbate 80.

The present invention also relates to a stable pharmaceutical formulation obtained by adding excipients to the antibody solution obtained by the process disclosed herein. The stable pharmaceutical formulation may be liquid, lyophilized or reconstituted. In one aspect of the present invention the pharmaceutical formulation is liquid.

A “liquid” formulation is one that has been prepared in a liquid format. Such a formulation may be suitable for direct administration to a subject or, alternatively, can be packaged for storage either in a liquid form, in a frozen state or in a dried form (e.g. lyophilized) for later reconstitution into a liquid form or other forms suitable for administration to a subject.

The term “buffer” as used herein refers to a buffered solution that resists changes in pH by the action of its acid-base conjugate components. A buffer of this invention has a pH in the range from about 5.0 to about 7.0; and preferably is 6.0±0.5. Examples of buffers that can control the pH in this range include acetate (e.g. sodium acetate), succinate (such as sodium succinate), gluconate, amino acids, such as histidine (e.g. histidine-HCl), citrate, phosphate, other organic acid buffer, their salts and combinations of buffers. In one embodiment of the present invention the buffer is present within the pharmaceutical formulation at concentration between about 1 mM and about 100 mM; in a more specific embodiment the concentration of the buffer is between about 5 mM and about 50 mM; preferably the concentration of the buffer is about 25 mM. In another aspect of the present invention, the concentration of the buffer is at least about 1 mM, at least about 5 mM, at least about 10 mM, at least about 20 mM, at least about 25 mM, at least about 30 mM, at least about 40 mM, at least about 50 mM, at least about 60 mM, at least about 70 mM, at least about 80 mM, at least about 90 mM, at least about 100 mM. The present invention also includes a buffer with a concentration at any intermediate value of the above said values. In a particular embodiment of the present invention, the buffer is Histidine, e.g. histidine-HCl, present within the pharmaceutical formulation at a concentration of about 25 mM; in another particular embodiment the buffer is citrate, present within the pharmaceutical formulation at a concentration of about 25 mM.

A stabilizing or tonicity agent may be added to the formulation to stabilize the protein in the lyophilized form. Said stabilizing or tonicity agent is selected from the group comprising sodium acetate, sodium bicarbonate, sodium carbonate, sodium chloride (NaCl), potassium acetate, potassium bicarbonate, potassium carbonate, potassium chloride, calcium chloride (CaCl2)) sugars such as sucrose, glucose and trehalose, polyols such as mannitol, maltitol, sorbitol, xylitol, erythritol, and isomalt, polyethylene glycol, such as PEG400, Ethylenediaminetetraacetic acid (EDTA), amino acids such as histidine (e.g. histidine-HCl), arginine (e.g. arginine hydrochloride) and glycine, methionine, proline, lysine (e.g. lysine-HCl), glutamic acid, glutamine, cysteine, amines, glutathione, cyclodextrin, such as such as Hydroxypropyl β-cyclodextrin (HPBCD), Hydroxypropyl-sulfobutyl β-cyclodextrin (HPSBCD), Sulfobutylether β-cyclodextrin (SBECD), β-cyclodextrin (BetaCD), α-cyclodextrin (Alpha CD) and γ-cyclodextrin (Gamm CD) and surfactants. Non limiting examples of a typical surfactant include: non-ionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), poly glycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxy ethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as Cio-Cis alkyl sulfates salts (e.g. sodium cetyl sulfate, sodium lauryl sulfate, sodium oleyl sulfate), polyoxyethylene Cio-Cis alkyl ether sulfates salts with an average of 2-4 moles of ethylene oxide (e.g. sodium polyoxyethylene lauryl sulfate), and Cs-Cis alkyl sulfosuccmate ester salts (e.g. sodium lauryl sulfosuccmate ester); natural surfactants such as lecithin, glycerophospho lipid, sphingophospho lipids (e.g. sphingomyelin) and sucrose esters of C12-C18 fatty acids; Poloxamers such as Poloxamer 188, Poloxamer 407, Poloxamer 124, Poloxamer 237, Poloxamer 338; salts and combinations of the above cited components.

In a certain aspect of the present invention, the stabilizing or tonicity agent is present within the pharmaceutical formulation at a concentration between about 0.001 mg/mL and about 300 mg/mL, i.e. between about 50 mg/mL and 100 mg/mL, between about 70 mg/mL and 200 mg/mL, between about 5 mg/mL and 50 mg/mL, between about 1 mg/mL and 20 mg/mL, between about 0.1 mg/mL and 10 mg/mL, between about 0.001 mg/mL and 0.1 mg/mL. In another aspect of the present invention, the stabilizing or tonicity agent is present within the pharmaceutical formulation at a concentration between about 1 mM and about 300 mM, i.e. between about 5 mM and 200 mM, specifically about 10 mM, or about 50 mM, or about 100 mM, or about 150 mM, or about 200 mM. In another aspect of the present invention, the stabilizing or tonicity agent is present within the pharmaceutical formulation at a concentration between about 0.001% and about 15%, i.e. between about 0.01% and about 5% or between about 0.03% and about 3%. In a particular aspect, the pharmaceutical formulation of the present invention comprises one or more stabilizing or tonicity agent(s) selected form the group comprising sodium chloride, arginine-HCl, proline, mannitol, lysine-HCl, sucrose, methionine and a surfactant. In a more particular aspect the pharmaceutical formulation of the present invention comprises NaCl present within the pharmaceutical formulation at a concentration between about 100 mM and about 200 mM, specifically between about 130 mM and about 170 mM, even more specifically NaCl is present within the pharmaceutical formulation at a concentration of about 150 mM. In another particular aspect the pharmaceutical formulation of the present invention comprises arginine-HCl present within the pharmaceutical formulation at a concentration between about 100 mM and about 200 mM, specifically between about 130 mM and about 170 mM, even more specifically arginine-HCl is present within the pharmaceutical formulation at a concentration of about 150 mM. In another particular aspect the pharmaceutical formulation of the present invention comprises proline present within the pharmaceutical formulation at a concentration between about 100 mM and about 200 mM, specifically between about 130 mM and about 170 mM, even more specifically proline is present within the pharmaceutical formulation at a concentration of about 150 mM. In another particular aspect the pharmaceutical formulation of the present invention comprises mannitol present within the pharmaceutical formulation at a concentration between about 0.5% and about 10%, specifically between about 1% and about 5%, even more specifically mannitol is present within the pharmaceutical formulation at a concentration of selected from the group comprising about 1%, about 2%, about 2.5%, about 3%, about 4%, about 4.5%. In another particular aspect the pharmaceutical formulation of the present invention comprises lysine-HCl present within the pharmaceutical formulation at a concentration between about 100 mM and about 200 mM, specifically between about 130 mM and about 170 mM, even more specifically lysine-HCl is present within the pharmaceutical formulation at a concentration of about 150 mM. In another particular aspect the pharmaceutical formulation of the present invention comprises sucrose present within the pharmaceutical formulation at a concentration between about 1% and about 15%, specifically between about 5% and about 10%, even more specifically sucrose is present within the pharmaceutical formulation at a concentration of about 8.5%. In another particular aspect the pharmaceutical formulation of the present invention comprises methionine present within the pharmaceutical formulation at a concentration between about 1 mM and about 50 mM, specifically between about 5 mM and about 25 mM, even more specifically methionine is present within the pharmaceutical formulation at a concentration of about 10 mM. In another aspect the pharmaceutical formulation of the present invention comprises a surfactant present within the pharmaceutical formulation at a concentration between about 0.001% and about 1%, specifically between about 0.005% and 0.5%, more specifically between about 0.01% and 0.1%, even more specifically the surfactant is present within the pharmaceutical formulation at a concentration selected from the group comprising about 0.03%, about 0.04%, about 0.05%, and about 0.1%. Preferably, the surfactant is selected from polyoxyethylene sorbitan fatty acid esters. Particularly preferably the surfactant is Polysorbate 20, 21, 40, 60, 65, 80, 81 and 85, most preferably Polysorbate 80. Polysorbate 80 is also known by the brand name Tween 80™ (ICI Americas, Inc.). In a specific embodiment of the present invention, the surfactant is Polysorbate 80, present within said pharmaceutical formulation at a concentration of about 0.036%, or about 0.054%, or about 0.1%. The present invention also includes a stabilizing or tonicity agent at any intermediate value of the above said values.

In a specific embodiment of the present application the stable pharmaceutical formulation comprises an a antibody or fragment thereof present within said pharmaceutical formulation at a concentration of about 150 g/L, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v).

The present invention also relates to a process of production of a bulk drug substance or a drug product comprising the steps of:
(a) Protein A chromatography of a clarified cell harvest comprising an antibody;
(b) Viral inactivation of the resulting protein A eluate;
(c) Neutralization of the protein A eluate to pH 5.2, followed by 0.2 μm filtration;
(d) Cation exchange chromatography of the neutralized protein A eluate, followed by 0.2 μm filtration;
(e) First UF/DF of the cation exchange chromatography eluate, followed by 0.2 μm filtration;
(f) Anion exchange chromatography in flow through mode performed by membrane adsorption, followed by 0.2 am filtration;
(g) Viral nanofiltration;
(h) Second UF/DF of the nanofiltrated solution, followed by 0.2 um filtration;
(i) Third UF/DF of the antibody solution obtained by the second UF/DF according to the processes for obtaining a highly concentrated antibody solution as disclosed herein, followed by 0.2 um filtration;
(j) Obtaining a stable pharmaceutical formulation by adding excipients to the highly concentrated antibody solution obtained by the third UF/DF, followed by 0.2 um filtration.

In one aspect of the present invention the antibody solution obtained by the disclosed process comprises an antibody or an antibody fragment thereof. The antibody or antibody fragment thereof may be a “humanized antibody”, namely an antibody in which CDR sequences derived from the germline of another mammalian species, such as a mouse, have been grafted onto human framework sequences, and where additional framework region modifications may be made within the human framework sequences as well as within the CDR sequences derived from the germline of another mammalian species.

The antibody in the highly concentrated solution obtained by the process of the present invention may be used to treat patients in need thereof by administration of a therapeutically effective amount.

A “patient” for the purposes of the present invention includes both humans and other animals, preferably mammals and most preferably humans. Thus the antibodies of the present invention have both human therapy and veterinary applications. The term “treatment” or “treating” in the present invention is meant to include therapeutic treatment, as well as prophylactic, or suppressive measures for a disease or disorder. Thus, for example, successful administration of an antibody prior to onset of the disease results in treatment of the disease. As another example, successful administration of an antibody after clinical manifestation of the disease to combat the symptoms of the disease comprises treatment of the disease.

“Treatment” and “treating” also encompasses administration of an antibody after the appearance of the disease in order to eradicate the disease. Successful administration of an antibody after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

The antibody or of the present invention can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, for example, intranasally, orally, vaginally, rectally, sublingually or topically.

The antibody of the present invention can be administered at a single or multiple doses. The term “dose” or “dosage” as used in the present invention are interchangeable and indicates an amount of drug substance administered per body weight of a subject or a total dose administered to a subject irrespective to their body weight.

Administration is preferably in a “therapeutically effective amount”, this being sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage etc, is within the responsibility of medical doctors. Appropriate doses of antibody are well known in the art (Ledermann J A et al., (1999) Int J Cancer 47: 659-664; Bagshawe K D et a/., (1991) Antibody, Immunoconjugates and Radiopharmaceuticals, 4: 915-922). The precise dose will depend upon a number of factors, including the size and location of the area to be treated, body weight of the subject, the precise nature of the antibody (e.g. whole antibody or fragment) and any additional therapeutic agents administered before, at the time of or after administration of the antibody. The therapeutically effective amount of the pharmaceutical formulation according to a particular aspect of the present invention is administrated subcutaneously using a prefilled syringe.

The antibody or antibody fragment thereof may be an agonist or an antagonist antibody or fragment thereof. In certain particular aspects of the present invention, the antibody or antibody fragment thereof is an antagonist antibody. In a more specific aspect the antibody or antibody fragment thereof in the solution obtained by the process disclosed herein is an anti-OX40 antagonist antibody or fragment thereof. In an even more specific aspect, the anti-OX40 antagonist antibody or fragment is ISB830 (CAS Registry Number 2126777-87-3).

The term “anti-OX40 antagonist antibody or fragment thereof” is used herein to indicate antibodies or antibody fragments thereof that bind to OX40 e.g. human OX40, and are capable of inhibiting and/or neutralising the biological signalling activity of OX40, for example by blocking binding or substantially reducing binding of OX40 to OX40 ligand and thus inhibiting or reducing the signalisation pathway triggered by OX40 and/or inhibiting or reducing an OX40-mediated cell response like lymphocyte proliferation, cytokine expression, or lymphocyte survival. The anti-OX40 antagonist antibody or fragment thereof may therefore be used in the treatment of patients suffering of an OX40-mediated disorders.

As used herein, the term “OX40-mediated disorder” includes conditions such as allergy, asthma, COPD, rheumatoid arthritis, psoriasis and diseases associated with autoimmunity and inflammation. In particular, according to the present invention, exemplary OX40 mediated disorders include infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, Lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

Other exemplary OX40 mediated disorder include infections (viral, bacterial, fungal and parasitic), endotoxic shock associated with infection, arthritis, rheumatoid arthritis, asthma, bronchitis, influenza, respiratory syncytial virus, pneumonia, chronic obstructive pulmonary disease (COPD), idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, Lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

Preferably, the anti-OX40 antagonist antibody is used for the treatment or prevention of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

The present invention also discloses a stable pharmaceutical formulation obtained by adding excipients to the anti-OX40 antibody solution obtained by the process disclosed herein.

In certain embodiment of the present invention, the anti-OX40 antibody is present in the pharmaceutical formulation at a concentration between about 1 mg/mL and about 200 mg/mL. In a specific embodiment the anti-OX40 antibody is present in the pharmaceutical formulation at a concentration between about 1 mg/mL and 100 mg/mL, more specifically at a concentration between about 5 mg/mL and about 50 mg/mL, more specifically at a concentration of about 10 mg/mL. The present invention also includes the anti-OX40 antibody with a concentration at any value between the above cited concentrations. According to another aspect of the present invention, the anti-OX40 antibody or fragment thereof is present within the pharmaceutical formulation at a concentration between about 100 mg/ml and about 200 mg/mL, specifically at a concentration between about 130 mg/ml and 180 mg/ml, more specifically at a concentration of about 150 mg/mL. In one aspect of the present invention, the concentration of anti-OX40 antibody or fragment thereof is selected from the group of at least about 1 mg/mL, at least about 10 mg/mL, at least about 100 mg/mL, at least about 130 mg/mL, at least about 150 mg/mL, at least about 170 mg/mL, at least about 200 mg/mL. The present invention also includes concentrations of an anti-OX40 antagonist antibody or fragment thereof at any intermediate value of the above said values. In a preferred embodiment the concentration of the anti-OX40 antibody or fragment thereof is about 150 mg/mL.

In one particular embodiment of the present invention, the pharmaceutical formulation comprises an anti-OX40 antagonist antibody or fragment thereof present within said pharmaceutical formulation at a concentration of about 10 mg/mL, histidine buffer present within said pharmaceutical formulation at a concentration of about 15 mM, sodium chloride present within said pharmaceutical formulation at a concentration of about 150 mM and Polysorbate 80 present within said pharmaceutical formulation at a concentration of about 0.01% (w/v), and said pharmaceutical formulation has pH of about 6.25.

In another particular embodiment of the present invention, the pharmaceutical formulation comprises an anti-OX40 antagonist antibody or fragment thereof present within said pharmaceutical formulation at a concentration between about 150 mg/mL and about 200 mg/mL, more specifically between about 160 mg/mL and about 195 mg/mL, or between about 160 mg/mL and about 175 mg/mL, histidine buffer present within said pharmaceutical formulation at a concentration of about 25 mM, sodium chloride or arginine-HCl present within said pharmaceutical formulation at a concentration of about 150 mM, and said pharmaceutical formulation has pH or about 6.0±1.

In another particular embodiment of the present invention, the pharmaceutical formulation comprises an anti-OX40 antagonist antibody or fragment thereof present within said pharmaceutical formulation at a concentration between about 150 mg/mL and about 200 mg/mL, more specifically between about 165 mg/mL and about 181 mg/mL, histidine-HCl buffer present within said pharmaceutical formulation at a concentration of about 25 mM, arginine-HCl present within said pharmaceutical formulation at a concentration of about 150 mM, and Polysorbate 80 present within said pharmaceutical formulation at a concentration of about 0.1% (w/v), and said pharmaceutical formulation has pH or about 6.0±1.

In another embodiment the pharmaceutical formulation comprises an anti-OX40 antagonist antibody or fragment thereof present within said pharmaceutical formulation at a concentration of about 150 mg/mL, an histidine-HCl buffer present within said pharmaceutical formulation at a concentration of about 25 mM, arginine-HCl or sodium chloride present within said pharmaceutical formulation at a concentration of about 150 mM, optionally methionine present within said pharmaceutical formulation at a concentration of about 10 mM, and Polysorbate 80 present within said pharmaceutical formulation at a concentration between about 0.03% (w/v) and about 0.06% (w/v), and wherein said pharmaceutical formulation has pH between about 5 and about 7.

In another embodiment the pharmaceutical formulation comprises an anti-OX40 antagonist antibody or fragment thereof present within said pharmaceutical formulation at a concentration of about 150 mg/mL, histidine HCl buffer present within said pharmaceutical formulation at a concentration of about 25 mM, mannitol present within said pharmaceutical formulation at a concentration between about 1% and about 5% or sucrose present within said pharmaceutical formulation at a concentration of about 8.5%, optionally proline or lysine-HCl present within said pharmaceutical formulation at a concentration of about 150 mM or arginine-HCl present within said pharmaceutical formulation at a concentration of about 50 mM, and Polysorbate 80 present within said pharmaceutical formulation at a concentration of about 0.036% (w/v), and wherein said pharmaceutical formulation has pH of about 6.

In another embodiment the pharmaceutical formulation comprises an anti-OX40 antagonist antibody or fragment thereof present within said pharmaceutical formulation at a concentration of about 150 mg/mL, citrate buffer present within said pharmaceutical formulation at a concentration of about 25 mM, sodium chloride or arginine-HCl present within said pharmaceutical formulation at a concentration of about 150 mM, and Polysorbate 80 present within said pharmaceutical formulation at a concentration of about 0.036% (w/v), and wherein said pharmaceutical formulation has pH of about 6.

In a more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, sodium chloride present within said pharmaceutical formulation at a concentration of about 150 mM and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v), and said pharmaceutical formulation has pH of about 5.5.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v), and said pharmaceutical formulation has pH of about 6.5.

In another more particular embodiment of the present invention, the pharmaceutical formulation comprises an anti-OX40 antagonist antibody or fragment thereof present within said pharmaceutical formulation at a concentration of about 150 mg/mL, citrate buffer present within said pharmaceutical formulation at a concentration of about 25 mM, sodium chloride present within said pharmaceutical formulation at a concentration of about 150 mM and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, citrate buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, proline present within said pharmaceutical formulation at a concentration of about 150 mM, mannitol present within said pharmaceutical formulation at a concentration of about 2.5% and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, lysine-HCl present within said pharmaceutical formulation at a concentration of about 150 mM, mannitol present within said pharmaceutical formulation at a concentration of about 1% and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 50 mM, mannitol present within said pharmaceutical formulation at a concentration of about 3% and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 150 mM, methionine present within said pharmaceutical formulation at a concentration of about 10 mM and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, mannitol present within said pharmaceutical formulation at a concentration of about 4.2% and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, sucrose present within said pharmaceutical formulation at a concentration of about 8.5% and Polysorbate 80 present within said pharmaceutical formulation at a concentration of about 0.036% (w/v), and said pharmaceutical formulation has pH of about 6.

In another more particular embodiment of the present invention, the pharmaceutical formulation comprises an anti-OX40 antagonist antibody or fragment thereof present within said pharmaceutical formulation at a concentration of about 150 mg/mL, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.054% (w/v), and said pharmaceutical formulation has pH of about 6.

The pharmaceutical formulation according to the present invention is stable. A “stable” formulation is one in which the protein therein essentially retains its physical stability and/or chemical stability and/or biological activity upon storage. Various analytical techniques for measuring protein stability are available in the art and are reviewed for example in Peptide and Protein Drug Delivery, 247-301, Vincent Lee Ed., Marcel Dekker, Inc., New York, N.Y., Pubs. (1991) and Jones A (1993) Adv Drug Delivery Rev, 10: 29-90. Stability can be measured at a selected temperature for a selected time period.

Analytical tests useful to determine said stability include but are not limited to: monitoring of the visual appearance as a significant change in the appearance of sample may indicate product degradation and/or microbial contamination leading to safety risk for the patients; sub-visible particles analysis, as the presence of higher sub-visible particles in parental solutions may lead to immunogenic responses; protein content measurement (e.g. by measuring absorbance at 280 nm wavelength (A280) by UV-VIS Spectroscopy or by SoloVPE) as any significant variation from its target concentration would not provide effective dose to patients; pH measurement as changes in pH may be indicative of degradation of buffering agents and lead to protein instability; size variants monitoring (e.g. by SE-HPLC and/or by cGE reduced and non-reduced) as changes in monomeric content toward aggregates (higher size than monomer) or fragments (smaller size than monomer) is an indication of its degradation; charge variants monitoring (e.g. by clEF) as changes in content of charged variants is an indication of its degradation; antibody potency measurement (e.g. by ELISA) as any significant change of binding property of the antibody toward its target would indicate antibody degradation. Additionally, the amino acid sequence as well as post-translational modifications (i.e. deamidation, oxidation, glycation, N-terminal variants, C-terminal variants and glycosylation site occupancy) can be verified, for instance by peptide mapping. Other characteristics of the formulation can be monitored, such as osmolarity and viscosity, as well as the protein thermal stability for instance by nano-format of Differential Scanning Fluorimetry (DSF), syringeability.

In one aspect of the present invention, the pharmaceutical formulation is stable under stresses comprising: rolling stress, storage at temperature higher than the room temperature, such as storage at +40° C. for at least 1 week; shaking such as shaking at 900 rpm for at least 24 hours, at least 48 hours, at least 72 hours at room temperature (25° C.); freezing-thawing circles such as freezing at −20° C. or at −80° C. and thawing at about 5° C., or at about 20° C., or at about 25° C., more in particular such as 3 to 5 freeze-thaw cycles by freezing at −80° C. and thawing at +5° C., such as 3 to 5 freeze-thaw cycles by freezing at −80° C. and thawing at +25° C., and such as freeze-thaw cycles by freezing at −20° C. and thawing at 20° C.

According to one aspect of the present invention, the pharmaceutical formulation is stable at temperature equal to or less than about 40° C., i.e. at a temperature between about −80° C. and 40° C., for at least about 1 month, or at least about 3 months, or at least about 6 months, or at least about 9 months, or at least about 12 months, or at least about 18 months, or at least about 24 months, or at least about 30 months, or at least about 36 months. The present invention also includes the disclosed pharmaceutical formulations stable at any value between the above cited temperatures, and at any time intervals between the above cited times. In one embodiment of the present invention, the pharmaceutical formulation of the present invention is stable when stored for at least about 1 month, or at least about 2 months, or at least about 3 months, or at least about 6 months, or at least about 9 months, or at least about 12 months, or at least about 18 months, or at least about 24 months, or at least about 30 months, or at least about 36 months, at a temperature of about of at least about 5° C., more specifically at a temperature of about 5° C. or at a temperature of about 25° C.; in a specific embodiment the pharmaceutical formulation of the present invention is stable when stored for at least about 9 month at a temperature of about 5° C. or at a temperature of about 25° C., for properties comprising glycation, sequence terminal modifications, and potency. In another specific aspect, the pharmaceutical formulation according to the present invention is stable when stored for at least about 36 months at a temperature of about 5° C. In a more specific embodiment, the disclosed pharmaceutical formulation is stable at about +25±2° C. for at least 1 month; in another specific embodiment, the disclosed pharmaceutical formulation is stable at about +5±3° C. for at least about 1 month, preferably for at least about 3 months; in another specific embodiment, the disclosed pharmaceutical formulation is stable at about −80±20° C. or about −20±5° C. for at least about 1 month, or at least about 3 months, preferably for at least about 6 months. In another more specific embodiment, the disclosed pharmaceutical formulation is stable at about 40±2° C. for at least about 1 month; in another specific embodiment, the disclosed pharmaceutical formulation is stable at about 25±2° C. for at least about 1 month, more specifically for least about 3 months, preferably for at least about 6 months; in another specific embodiment, the disclosed pharmaceutical formulation is stable at about 5±3° C. for at least about 1 month, specifically for at least about 3 months, more specifically for at least about 6 months, even more specifically for at least about 12 months, preferably for at least about 18 months, more preferably for at least about 24 months.

In a more particular embodiment the pharmaceutical formulation comprising an anti-OX40 antagonist antibody or fragment thereof present within said pharmaceutical formulation at a concentration of about 150 mg/mL, an histidine-HCl buffer present within said pharmaceutical formulation at a concentration of about 25 mM, proline or sodium chloride present within said pharmaceutical formulation at a concentration of about 150 mM, or arginine-HCl present within said pharmaceutical formulation at a concentration between about 50 mM and about 150 mM, optionally methionine present within said pharmaceutical formulation at a concentration of about 10 mM of mannitol present within said pharmaceutical formulation at a concentration between about 2% and about 3.5%, and Polysorbate 80 present within said pharmaceutical formulation at a concentration between about 0.03% and about 0.06% (w/v), and having pH between about 5 and about 7, is stable for at least about 6 months at a temperature of about 25° C. or less.

In another more particular embodiment the pharmaceutical formulation comprising an anti-OX40 antagonist antibody or fragment thereof present within said pharmaceutical formulation at a concentration of about 150 mg/mL, histidine HCl buffer present within said pharmaceutical formulation at a concentration of about 25 mM, arginine-HCl or sodium chloride present within said pharmaceutical formulation at a concentration of about 150 mM and Polysorbate 80 present within said pharmaceutical formulation at a concentration of about 0.036% (w/v), and having pH of about 6 is stable for at least about 18 months at a temperature of about 5° C. or less.

The liquid formulation of the present invention can be comprised within a container. For instance a container made of glass, plastic, other polymers. Non limiting examples of said container are syringes, such as pre-filled syringe, glass cartridge syringe, autoinjectors, bottles, vials, and test tubes, pens, bags such as storage bags. The liquid formulation according to the present invention can be contained in a prefilled syringe for subcutaneous injection using an autoinjectors.

In one embodiment of the present invention, the pharmaceutical formulation is comprised within a glass vial, such as a 2 mL glass vials stoppered with 13 mm injection rubber stopper or such as a 10 mL glass vial stoppered with a 20 mm injection flurotec-coated stopper.

In another embodiment of the present invention the pharmaceutical formulation is comprised within a prefilled syringe (PFS). A PFS may be filled with a volume such as about 0.1 mL, or about 0.2 mL, or about 0.5 mL, or about 1 mL, or about 1.5 mL, or about 2 mL, or about 2.5 mL, or about 3 mL of the formulation according to the present invention. In particular, the prefilled syringe comprises a glass barrel characterized by a needle having gauge between 25 and 30 G and a siliconization level between 0.2 mg and 1 mg per prefilled syringe, and a halobutyl stopper; wherein the needle may be a staked needle or a needle externally mounted for instance on a Luer lock of a syringe tip. Suitable material of which the PFS is made include and are not limited to glass and plastic. In a specific embodiment about 2 mL, such as 2.1 mL of said pharmaceutical formulation is filled in 2.25 mL glass pre-fillable syringe which comprises a glass barrel characterized by a staked needle having gauge of about 27 G 1/2″ and a siliconization level between 0.3 mg and 0.7 mg per prefilled syringe, and a halobutyl stopper. Examples of commercially available pre-filled syringe barrels/stoppers are Gerresheimer, Schott, OMPI, BD NovaPure, West Pharmaceutical Services.

To determine the stability of the prefilled syringe filled with the formulation according to the present invention, the analytical tests mentioned above are applicable. Additionally, the performance of syringe (i.e. Syringeability) may be judged based on two forces namely, the force required to initiate plunger movement (break-loose force) and the force required to maintain the movement of plunger (gliding force). Any significant change in these two forces may indicate poor syringe performance which may lead to inaccuracy in dose volume and/or pain to patients during injection.

In one aspect of the present invention, the pharmaceutical formulation is expelled from the PFS in a time between 5 s and 30 s, preferably in a time between 10 s and 20 s, more preferably in 15 s. In another aspect of the present invention, when the pharmaceutical formulation is expelled from the PFS in a time between 10 s and 20 s, the break lose force is between about 2N and about 4N and the gliding force is between about 4N and about 30N, for instance between about 4N and 13N.

According to one aspect of the present invention, the pharmaceutical formulation comprised within a 2.25 mL glass prefilled syringe is stable at temperature equal to or less than about 40° C., i.e. at a temperature between about −80° C. and 40° C., for at least about 1 month, or at least about 3 months, or at least about 6 months, or at least about 9 months, or at least about 12 months, or at least about 18 months, or at least about 24 months. The present invention also includes the disclosed pharmaceutical formulation stable at any value between the above cited temperatures, and at any time intervals between the above cited times. In a more specific embodiment, the disclosed pharmaceutical formulation is stable at about 40±2° C. for at least about 1 month; in another specific embodiment, the disclosed pharmaceutical formulation is stable at about 25±2° C. for at least about 1 month, more specifically for least about 3 months; in another specific embodiment, the disclosed pharmaceutical formulation is stable at about 5±3° C. for at least about 1 month, specifically for at least about 3 months, more specifically for at least about 6 months, even more specifically for at least about 12 months, preferably for at least about 18 months, more preferably for at least about 24 months.

In a more particular embodiment the pharmaceutical formulation comprising an anti-OX40 antagonist antibody or fragment thereof present within said pharmaceutical formulation at a concentration of about 150 mg/mL, an histidine-HCl buffer present within said pharmaceutical formulation at a concentration of about 25 mM, proline or sodium chloride present within said pharmaceutical formulation at a concentration of about 150 mM, or arginine-HCl present within said pharmaceutical formulation at a concentration between about 50 mM and about 150 mM, optionally methionine present within said pharmaceutical formulation at a concentration of about 10 mM or mannitol present within said pharmaceutical formulation at a concentration between about 2% and about 3.5%, and Polysorbate 80 present within said pharmaceutical formulation at a concentration between about 0.03% and about 0.06% (w/v), and having pH between about 5 and about 7, is comprised within a prefilled syringe and it is stable for at least about 6 months at a temperature of about 25° C. or less. In another more particular embodiment the pharmaceutical formulation comprising an anti-OX40 antagonist antibody or fragment thereof present within said pharmaceutical formulation at a concentration of about 150 mg/mL, histidine HCl buffer present within said pharmaceutical formulation at a concentration of about 25 mM, arginine-HCl or sodium chloride present within said pharmaceutical formulation at a concentration of about 150 mM and Polysorbate 80 present within said pharmaceutical formulation at a concentration of about 0.036% (w/v), and having pH of about 6, is comprised within a prefilled syringe and it is stable for at least about 18 months at a temperature of about 5° C. or less.

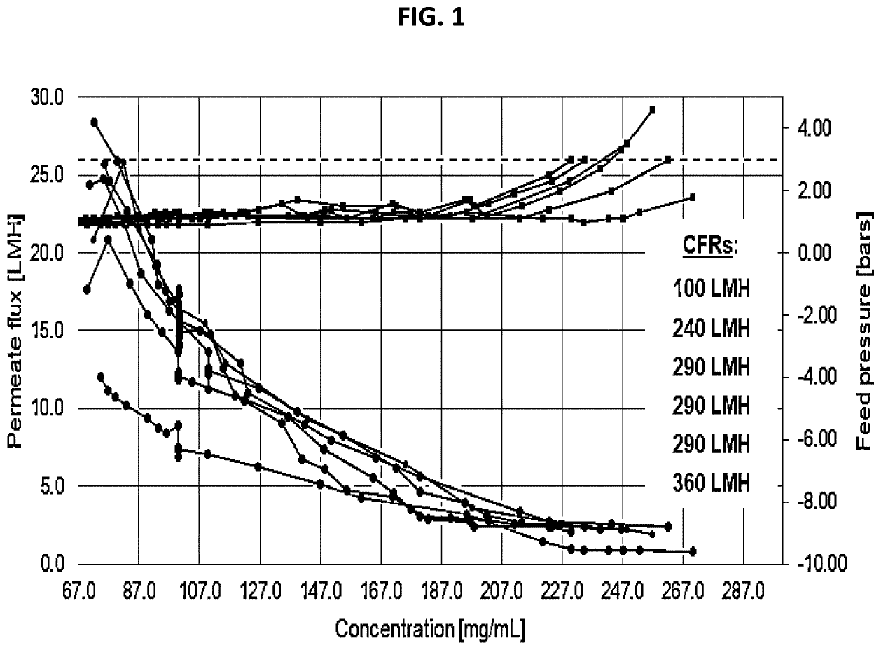

FIG. 1: UFDF 3 process performance (permeate flux/TMP vs. concentration)

### EXAMPLES

### Example 1: Concentration of an Antibody Solution

### Downstream Process (DSP)

To develop a process for obtaining a highly concentrated antibody solution, first a cell culture harvest obtained from 12 to 14 days culture of CHO cells expressing the antibody Ab1 was clarified and subjected to the following downstream processing steps.

First the clarified cell harvest was subjected to a protein A chromatography following standard procedure, next the obtained protein A chromatography eluate was subjected to a viral inactivation step performed by holding the solution at pH 3.5±0.1, for 60 minutes. The viral inactivation was then neutralized to pH 5.2, and followed by a depth and a 0.2 μm filtration. Next, cation exchange chromatography (CEX) was carried out in a bind-eluate where the antibody was eluted with a 25 mM Citrate, 100 mM NaCl pH 5.2 elution buffer, and was followed by a 0.2 μm filtration. In order to concentrate antibody in the CEX eluate to 20 g/L a first ultrafiltration/diafiltration (UFDF-1) step was carried out using a tangential flow filtration cassette with a nominal molecular weight limit (NMWL) of 30 kDa, a diafiltration buffer comprising 50 mM Histidine at pH 6.5 was used for this step. UFDF-1 was followed by a 0.2 μm filtration. Next anion exchange chromatography was carried out by membrane absorption (MA) in flow-through mode using 50 mM Histidine pH 6.5 buffer, and was followed first by a 0.2 μm filtration, next by a virus nanofiltration carried out using 50 mM Histidine pH 6.5. A second UFDF (UFDF-2) was then used to concentrate the obtained antibody solution to 70 g/L. A 5 mM L-Histidine buffer at pH 6.0 was used for the UFDF-2 step.

All the described steps were performed according to the provider recommendation. As we targeted a higher concentration of the antibody upon the purification process, we developed a further concentration step.

In particular we developed a third UFDF (UFDF-3) wherein starting from an antibody solution with an antibody concentration between about 60 to 80 g/L, it is possible to reach a higher target concentration such as 170 g/L. The developed UFDF is described in the next sections.

### UFDF-3 for High Concentration of and Antibody Solution—Conditions Selection

A UFDF-3 step has been developed to concentrate the product and reach the target concentration of 170 g/L after flushing, starting from an antibody solution with an antibody concentration between about 65 and about 75 g/L. The concentration was performed by Tangential Flow Filtration (TFF).

### UFDF Load and Product Storage Conditions

Freeze/thaw and hold time studies were performed on UFDF-3 load and product as described in Table 1:

TABLE 1
Hold time study conditions for Abl UFDF-3 product

| | Timepoints | | |
|---|---|---|---|
| Storage | T 0 | 1 week | 2 weeks |
| +5 ± 3° C. | x | x | x |
| +22.5 ± 2.5° C. | | x | x |

Thus UFDF 3 load and product were firstly frozen at −20° C., then thaw at room temperature and to finish re-frozen. UFDF-3 loads and products were analyzed by SE-HPLC, CGE (reduced and non-reduced), iCE and CEX-HPLC to assess the product quality. pH, conductivity, concentration and UFDF 3 loads and products osmolality were also measured.

### Results and Discussion

The selected cassette for the UFDF-3 step is a tangential flow filtration (TFF) cassette with nominal molecular weight limit (NMWL) of 30 KDa, named Cassette 1. The diafiltration was done using 25 mM Histidine, 150 mM Arginine pH 6.0 (DF buffer) during 7 DVs. The first concentration was made up to 100 g/L prior to be diafiltered into the aforementioned pre-formulation buffer. Then, the diafiltered product was concentrated again until the feed pressure reaches 3 bars (˜ 220 g/L). Even if higher pressures (until 5 bars) did not show an impact on product quality, the 3 bars maximum pressure corresponds to the limit of the TFF system (tubing limitation) according to recommendation.

This choice leads to certain constraints which were taken into account during development. They are described in the Table 2.

TABLE 2
Equipment characteristics and associated operating parameters
to consider during the UFDF-3 development

| System characteristics taken into account | Operating parameters to consider |
|---|---|
| Holder capacity | Cassettes surface |
| Feed pump maximum capacity | CFR selection |
| Retentate tank maximum capacity | Diafiltration concentration start |
| Retentate tank minimum capacity | Minimum load volume |
| Hold-up volume | Flush and minimum target pre-flushed |
| | concentration |
| Pressure | Process pressures (especially feed pressure) |

### Cross Flow Rate (CFR)

CFR is calculated using feed and permeate flow rates (CFR=Feed flow rate−Permeate flow rate). For this reason, a pump calibration was performed prior to this run (not shown) in order to ensure an accurate measurement of CFR. CFRs (from 100 LMH to 360 LMH), FIG. 1 shows the UFDF-3 process performances (permeate flux and TMP evolution throughout the UFDF-3 step) obtained with different CFRs (from 100 LMH to 360 LMH). As demonstrated, increasing the CFR tends to improve the permeate flux. Indeed the starting permeate flux obtained with 100 LMH CFR is approximately 13 LMH compared to that obtained with 360 LMH CFR which is approximately 25 LMH. Nevertheless, no significant permeate flux improvement from 240 LMH to 360 LMH CFRs is observed (˜ 20 LMH and 25 LMH for respectively 240 LMH and 360 LMH CFRs).

Regarding the pressure, a pressure drop is observed at the end of concentration. For runs performed with a CFR comprised between 240 and 360 LMH CFRs, this phenomenon occurs around 210 g/L whereas for lower CFR (^{˜}100 LMH), it appears later at about 250 g/L. However, even if there is an earlier pressure drop for CFR>100 LMH, working with higher CFR (>240 LMH) allows twice higher permeate flux, at the beginning and during diafiltration, thus allowing much lower process duration (gain of more than 2 hours of process time). Moreover, the reached pre-flushed concentration of 210 g/L is sufficient for a flush of 3 HUV. Consequently, a CFR range of ≥240 LMH-≤360 LMH is the best compromise to reach highest flux without having too much pressure.

### Maximum Feed Pump

The flow rate selection must take into account the maximum feed pump speed capacity of the selected TFF skid. Indeed, as the CFR is expressed in L/h per square meter, it is dependent on the cassette surface. Consequently, depending of the cassette surface used for a run, there is a risk to be above the maximum limit of the feed pump speed (i.e. 1000 L/h). The maximum feed pump (L/h) is the multiplication of the maximum feed flow rate (LMH) and the maximum surface area (m²):

$$Maximum feed pump ( L \text{/} h ) = Maximum feed flow rate ( LMH ) * Maximum surface ( {m}^{2} )$$

$$And consequently \text{:}$$

$$Maximum feed flow rate ( LMH ) = \frac{Maximum feed pump ( L \text{/} h )}{Maximum surface ( {m}^{2} )}$$

$$Considering a maximum holder capacity of 2.85 {m}^{2} , \\ the maximum feed flow rate calculation is \text{:}$$

$$Maximum feed flow rate ( LMH ) = \frac{Maximum feed pump ( L \text{/} h )}{Maximum surface ( {m}^{2} )} = \frac{1000 L \text{/} h}{2.85 {m}^{2}} = 351 LMH$$

Knowing that, at this feed flow rate (FFR), the permeate flow rate (PFR) is about 25 LMH, it means that the CFR would be:

CFR (LMH)=FFR (LMH)−PFR (LMH)=351 LMH−25 LMH=326 LMH

Consequently, a maximum CFR limit of 326 LMH should be established.

Moreover, to avoid working at the maximum of the pump which is not desirable, a set point at 290 LMH was defined. Based on small scale runs data, the permeate flow rate obtained at the beginning of the concentration with a CFR of 290 LMH was 25 LMH. Considering a maximum total surface area of 2.85 m², the associated feed flow rate for the defined CFR set point would be:

FFR (LMH)=CFR (LMH)+PFR (LMH)=290 LMH+25 LMH=315 LMH

Which corresponds to a feed pump speed of:

$$Feed pump speed ( L \text{/} h ) = FF R ( L M H ) \times max cassettes surface ( {m}^{2} ) = 315 LMH \times 2.85 {m}^{2} = 898 L \text{/} h$$

As the cassettes surface of 2.85 m² is the maximum stood for the manufacturing TFF system, the feed pump would speed is still suitable whatever the surface area used. Furthermore the maximum pump speed will never be reached. Thus the recommendation for the CFR is ≥240 LMH−≤325 LMH with a set point at 290 LMH.

### Volumetric Loading Factor (VLF)

The tested VLFs are described in the Table 3:

TABLE 3
Ab1 UFDF-3 VLF evaluation

| Parameters | Sample A | Sample B | Sample C | Sample D | Sample E |
|---|---|---|---|---|---|
| CFR [LMH] | 360 | 240 | 290 | 290 | 290 |
| Volumetric loading factor [L/m²] | 15 | 20 | 25 | 30 | 25 |
| UFDF-3 process duration [hours] | 5.2 | 9.4 | 9.8 | 11.8 | 9.3 |

Table 3 shows that higher the VLF is, longer is the process duration: 5 hours at 15 L/m², 9 hours at 20 L/m², and 11.8 hours at 30 L/m². According to SBL mass balance for a 5000 L batch, the highest expected UFDF-3 load volume would be 75 L. If the maximum VLF is set at 25 L/m², a cassette surface of 3 m² would be required which is not suitable for the maximum capacity of the holder (2×1.14 m2+1×0.57 m²). Therefore, the VLF of 30 L/m² was chosen as the upper limit to fit with potential highest volumes. Moreover, no product quality impact was noticed whatever the VLF. Thus the recommendation for the VLF is 30 L/m² with a set point at 25 L/m².

### Diafiltration

The scope of this experiment was to define the most appropriate product concentration to start the diafiltration. The choice was made on a compromise between sufficient volume reduction (to fit with the retentate tank volume capacity) while keeping the lowest DF duration (i.e. high permeate flux).

Previous experiments allowed to select a start of the DF for a product concentration of 100 g/L. Targeting this concentration allowed to put the product into the diafiltration buffer (25 mM L-Histidine, 150 mM L-Arginine-HCl pH 6.0) before reaching too high pressure caused by the increasing viscosity. Indeed, the diafiltration buffer allows a reduction of viscosity which is high when the product is in the equilibration buffer (5 mM L-Histidine pH 6.0).

According to SBL mass balance, for a 5000 L batch, the UFDF-3 load volume and concentration upper limits are 75 L at 75 g/L. Targeting 100 g/L for the diafiltration would lead to a DF product volume of 56.3 L which is higher than the maximum volume recommended by the supplier (55 L for the selected retentate tank of 50 L). Consequently, target DF concentration was optimized to mitigate this volume constraint. The results are presented in Table 4.

TABLE 4
Ab1 UFDF-3 DF concentration evaluation.

| Parameters | Sample C | Sample E |
|---|---|---|
| CFR [LMH] | 290 | 290 |
| Volumetric loading factor [L/m²] | 25 | 25 |
| Diafiltration concentration [g/L] | 110 | 100 |
| UFDF-3 process duration [hours] | 9.8 | 9.3 |

The only difference observed is the process duration which is slightly longer for a DF performed at 110 g/L compared to one performed at 100 g/L (9.8 hours than 9.3 hours respectively). However, this difference can be considered as negligible and no impact on product quality was observed. Thus, for high starting volumes, the recommendation is to start the diafiltration at a product concentration of 110 g/L. If this is not the case, targeting 100 g/L is reducing the whole process duration. The recommendation for the DF concentration is 110 g/L with a set point at 100 g/L. The actual value to target should be calculated while taking into account the initial volume and the retentate tank capacity.

### Worst Case Scenario 1: Initial Volume Upper Limit

Following the recommendation mentioned in the previous section, calculations were made to indicate which concentration to choose for different initial volumes (Table 5).

TABLE 5
DF concentration recommendation
for different UFDF-3 load volumes

| Initial volumes | | 70 L | 75 L | 80 L | 85.5 L |
|---|---|---|---|---|---|
| Cassette surface | | 2 × 1.14 m² + 1 × 0.57 m² | | | |
| Initial concentration | | 75 g/L | | | |
| VLF | | 25 L/m² | 26 L/m² | 28 L/m² | 30 L/m² |
| DF volumes | @ 100 g/L | 51.3 L | 55.1 L | 58.8 L | 62.9 L |
| | @ 110 g/L | 46.5 L | 49.9 L | 53.3 L | 57.1 L |

According to the scale up calculation provided above and in order to fit with the maximum retentate tank capacity of 55 L, it appears that the diafiltration step must be performed at 100 g/L up to 70 L initial volume. Above 70 L, the diafiltration has to be performed at 110 g/L.

### Worst Case Scenario 2: Initial Volume Lower Limit

In the case of lowest initial volume, the manufacturing constraint is the minimum working volume of the system. Indeed, the risk is to have a product pre-flushed volume below the minimum working volume which may lead to increase in foam and shear stress.

Therefore, an initial volume lower limit must be established to avoid having a pre-flushed volume below the minimum recommended recirculation volume. Table 6 shows estimated pre-flushed volumes for different potential initial volumes and assuming that the pre-flushed concentration is 220 g/L.

TABLE 6
UFDF-3 volumes estimation related to low initial volumes and concentration

| Initial volume | 40 L | 45 L | 50 L | 60 L |
|---|---|---|---|---|
| Cassette surface | 1 × 1.14 m2 + 1 × 0.57 m2 | | 2 × 1.14 m2 | |
| Initial concentration | 65 g/L | | | |
| VLF | 23 L/m2 | 29 L/m2 | 22 L/m2 | 26 L/m2 |
| Pre-flushed concentration | 220 g/L | | | |
| Pre-flushed volume | 10.9 L | 12.4 L | 13.7 L | 16.7 L |

According to the scale up calculation provided above, it appears that processing low initial volumes can be risky in term of the minimum retentate tank capacity and pre-flushed volumes achieved. This point need to be considered and hence determine before running step at scale. The recommendation here would be not to processed less than 40 L. Initial volumes able to be processed during this UFDF-3 step as well as the DF concentration are:
≥40 L-<70 L with a diafiltration performed at 100 g/L (maximum tank capacity)
≥70 L-≤80 L with a diafiltration performed at 110 g/L (minimum tank capacity).

### Product Quality

No impact on product quality has been observed during UFDF-3 development.

Table 7 shows two examples of product quality results obtained at the selected optimized conditions:

TABLE 7
UFDF-3 analytical results

| | Run | | Sample C | Sample D | Sample F |
|---|---|---|---|---|---|
| | Scale | | Small | | Pilot |
| LOAD | PH | | 6.6 | 6.6 | 6.5 |
| | Conductivity [mS/cm] | | 0.5 | 0.5 | 0.5 |
| | Concentration [g/L] | | 71.3 | | 67.3 |
| | SE-HPLC | Monomer [%] | 98.4 | 98.4 | 97.7 |
| | Non-reduced | IgG [%] | 92.1 | 93.4 | 92.5 |
| | CGE | Assigned peaks [%] | 6.6 | 6.5 | 7.0 |
| | Reduced CGE | LC [%] | 34.2 | 34.2 | 34.2 |
| | | HC [%] | 65.2 | 65.2 | 65.0 |
| | | Other peaks [%] | 0.7 | 0.6 | 0.9 |
| | cIEF | Acidic [%] | 29.6 | 30.3 | 27.0 |
| | | Main [%] | 47.8 | 47.1 | 48.6 |
| | | Basic [%] | 22.6 | 22.6 | 24.4 |
| | CEX-HPLC | Acidic [%] | 17.7 | 17.6 | 19.7 |
| | | Main [%] | 48.3 | 48.1 | 46.1 |
| | | Basic [%] | 34.0 | 34.3 | 34.2 |
| | Density [g/cm³] | | 1.0185 | 1.0186 | 1.0183 |
| | Viscosity [mPa/s] | | 3.405 | 2.292 | 2.708 |
| | Osmolality [mosm/kg] | | 10 | 12 | 11 |
| Quantity L/m² membrane | | | 25 | 30 | 20 |
| Quantity mAb/m² membrane | | | 1783 | 2139 | 1356 |
| Tested parameters | | CFR [LMH] | 290 | 290 | 290 |
| | | VLF [L/m²] | 25 | 30 | 20 |
| | | DF concentration [g/L] | 110 | 100 | 100 |
| PRODUCT | PH | | 6.0 | 6.1 | 6.1 |
| | Conductivity [mS/cm] | | 8.2 | 8.0 | 7.8 |
| | Concentration [g/L] | | 172.5 | 174.5 | 177.9 |
| | Flush [HUV] | | 3.5 | 2.8 | 3.2 |
| | SE-HPLC | Monomer [%] | 98.5 | 98.6 | 98.3 |
| | Non-reduced | IgG [%] | 92.3 | 92.8 | 91.9 |
| | CGE | Assigned peaks [%] | 6.5 | 6.6 | 7.6 |
| | Reduced CGE | LC [%] | 34.2 | 34.1 | 34.0 |
| | | HC [%] | 65.2 | 65.3 | 64.8 |
| | | Other peaks [%] | 0.6 | 0.7 | 1.2 |
| | cIEF | Acidic [%] | 29.4 | 29.4 | 26.9 |
| | | Main [%] | 47.8 | 48.0 | 49.1 |
| | | Basic [%] | 22.8 | 22.5 | 24.1 |
| | CEX-HPLC | Acidic [%] | 18.0 | 17.8 | 19.4 |
| | | Main [%] | 47.9 | 48.1 | 46.6 |
| | | Basic [%] | 34.2 | 34.1 | 34.0 |

| | | | |
|---|---|---|---|
| Density [g/cm³] | 1.0583 | 1.0623 | 1.0626 |
| Viscosity [mPa/s] | 10.61 | 13.45 | 15.43 |
| Osmolality [mosm/kg] | 317 | 339 | 325 |

| | | | |
|---|---|---|---|
| Yield [%] | 98 | 93 | 97 |

High viscosity observed for Sample C UFDF-3 load is unexpected. However, high deviation level (23%) occurred during the analysis. The yield obtained for Sample D (93%) is slightly lower than expected but it is easily explained by the lack of flush (2.8 HUV), lower than the specification set (≥3 HUV). Indeed the flush is an important point to consider, especially for such high concentrations, in order to recover as much product as possible. No significant differences were observed with respect to SE-HPLC (loads and products ≥98%) and CGE (loads and products ≥92% with less than 1% differences in all conditions). Regarding charged variant profiles by clEF, differences of about 1.5% were observed between small and pilot runs. No significant differences between respective load and product were detected, indicating that UFDF-3 does not charged variant profiles.

### Stability Studies

During the first assessment hold time and freeze and thaw studies have been performed using UFDF-3 load and product from Sample G. For each sample (load and product), all tested conditions were analyzed within the same sequence in order to minimize the variability. Table 8 and Table 9 represent hold time study results of respectively UFDF-3 load and product at different time points, namely 1 week (w) and 2 weeks, and storage temperatures.

TABLE 8
UFDF-3 load hold time study

| | UFDF-3 LOAD Sample G | | | | | |
|---|---|---|---|---|---|---|
| | | | +22.5 ± 2.5° C. | | +5 ± 3° C. | |
| | Conditions | T 0 | 1 w | 2 w | 1 w | 2 w |
| SE-HPLC | Aggregate [%] | 2.3 | 2.2 | 2.1 | 2.2 | 2.0 |
| | Monomer + Tailing | 97.6 | 97.7 | 97.8 | 97.7 | 97.9 |
| | [%] | | | | | |
| | Fragment [%] | 0.1 | 0.1 | 0.1 | 0.1 | 0.1 |
| Non-reduced | LC [%] | 1.8 | 1.7 | 1.6 | 1.7 | 1.7 |
| CGE | HC [%] | 0.1 | 0.1 | 0.1 | 0.1 | 0.2 |
| | 75 kD [%] | 0.0 | 0.1 | 0.1 | 0.0 | 0.0 |
| | 100 kD [%] | 0.6 | 0.5 | 0.7 | 0.6 | 0.7 |
| | 125 kD [%] | 4.7 | 4.8 | 4.9 | 4.6 | 5.0 |
| | IgG′ [%] | 2.4 | 2.7 | 3.3 | 2.8 | 2.7 |
| | IgG [%] | 90.3 | 90.1 | 89.3 | 90.2 | 89.9 |
| | Total IgG [%] | 92.7 | 92.8 | 92.6 | 93.0 | 92.6 |
| Reduced CGE | LC [%] | 38.2 | 38.2 | 37.8 | 38.3 | 38.7 |
| | HC aglycosyl [%] | 0.5 | 0.5 | 0.6 | 0.0 | 0.0 |
| | HC [%] | 61.3 | 61.3 | 61.6 | 61.7 | 61.3 |
| cIEF | Acidic [%] | 29.5 | 29.9 | 30.5 | 29.4 | 29.4 |
| | Main [%] | 49.7 | 49.0 | 48.8 | 49.2 | 49.2 |
| | Basic [%] | 20.8 | 21.1 | 20.7 | 21.4 | 21.4 |
| CEX-HPLC | Acidic [%] | 14.7 | 15.0 | 15.3 | 14.8 | 14.8 |
| | Main [%] | 56.5 | 56.2 | 56.4 | 56.6 | 56.4 |
| | Basic [%] | 28.8 | 28.8 | 28.3 | 28.7 | 28.8 |

TABLE 9
UFDF-3 product hold time study

| | UFDF-3 PRODUCT Sample G | | | | | |
|---|---|---|---|---|---|---|
| | | | +22.5 ± 2.5° C. | | +5 ± 3° C. | |
| | Conditions | T 0 | 1 w | 2 w | 1 w | 2 w |
| SE-HPLC | Aggregate [%] | 1.6 | 1.5 | 1.5 | 1.5 | 1.5 |
| | Monomer + | 98.4 | 98.5 | 98.4 | 98.4 | 98.4 |
| | Tailing [%] | | | | | |
| | Fragment [%] | 0.1 | 0.1 | 0.1 | 0.1 | 0.1 |
| Non-reduced | LC [%] | 1.8 | 1.8 | 1.7 | 1.8 | 1.8 |
| CGE | HC [%] | 0.1 | 0.1 | 0.1 | 0.0 | 0.1 |
| | 75 kD [%] | 0.0 | 0.1 | 0.0 | 0.1 | 0.1 |
| | 100 kD [%] | 0.7 | 0.7 | 0.7 | 0.8 | 0.7 |
| | 125 kD [%] | 4.9 | 5.3 | 5.1 | 5.4 | 5.4 |
| | IgG′ [%] | 2.9 | 2.9 | 3.1 | 3.4 | 3.0 |
| | IgG [%] | 89.6 | 89.3 | 89.2 | 88.6 | 89.0 |
| | Total IgG [%] | 92.5 | 92.2 | 92.3 | 92.0 | 92.0 |
| Reduced CGE | LC [%] | 39.0 | 38.6 | 38.8 | 37.4 | 37.4 |
| | HC aglycosyl [%] | 0.0 | 0.4 | 0.3 | 0.4 | 0.7 |
| | HC [%] | 61.0 | 61.0 | 60.9 | 62.1 | 61.8 |
| cIEF | Acidic [%] | 29.5 | 28.8 | 29.1 | 29.0 | 29.3 |
| | Main [%] | 49.3 | 49.7 | 49.7 | 49.7 | 49.5 |
| | Basic [%] | 21.2 | 21.6 | 21.2 | 21.1 | 21.2 |
| CEX-HPLC | Acidic [%] | 14.4 | 14.3 | 14.5 | 14.5 | 14.5 |
| | Main [%] | 56.8 | 56.5 | 56.4 | 56.4 | 56.5 |
| | Basic [%] | 28.8 | 29.2 | 29.1 | 29.1 | 29.1 |

Whatever the sample (load or product), no significant differences were detected regarding charge variant profiles neither by clEF nor by CEX-HPLC. SE-HPLC are all within method variability (≤0.3%) as well, observed variations are thus not significant. Slight variations observed with respect to CGE (reduced and non-reduced) are inherent to the method and thus results are considered as comparable. To conclude, UFDF-3 load and product are both stable up to 2 weeks at room temperature (+22.5±2.5° C.) as well as at +5±3° C.

### Freeze and Thaw Study

As said before, all tested conditions were analysed within the same sequence in order to minimize the variability. Table 10 represents freeze and thaw study results of the UFDF-3 product.

TABLE 10
UFDF-3 product freeze and thaw study

| | UFDF-3 PRODUCT Sample G | | | |
|---|---|---|---|---|
| | | −20° C. | | |
| | Conditions | 1 F/T | 2 F/T | 3 F/T |
| SE-HPLC | Aggregate [%] | 1.6 | 1.6 | 1.6 |
| | Monomer + Tailing [%] | 98.4 | 98.3 | 98.3 |
| | Fragment [%] | 0.1 | 0.1 | 0.1 |
| Non-reduced CGE | LC [%] | 1.5 | 1.8 | 1.7 |
| | HC [%] | 0.1 | 0.1 | 0.1 |
| | 75 kD [%] | 0.0 | 0.0 | 0.0 |
| | 100 kD [%] | 0.6 | 0.5 | 1.0 |
| | 125 kD [%] | 5.2 | 5.1 | 5.0 |
| | IgG' [%] | 2.5 | 3.2 | 3.0 |
| | IgG [%] | 90.0 | 89.3 | 89.3 |
| | Total IgG [%] | 92.5 | 92.5 | 92.3 |
| Reduced CGE | LC [%] | 35.8 | 35.6 | 36.2 |
| | HC aglycosyl [%] | 0.7 | 0.6 | 0.7 |
| | HC [%] | 62.5 | 62.7 | 62.1 |
| cIEF | Acidic [%] | 29.3 | 29.7 | 29.4 |
| | Main [%] | 49.7 | 49.1 | 49.5 |
| | Basic [%] | 21.1 | 21.1 | 21.1 |
| CEX-HPLC | Acidic [%] | 14.3 | 14.4 | 14.1 |
| | Main [%] | 56.9 | 56.8 | 56.9 |
| | Basic [%] | 28.8 | 28.8 | 28.9 |

There are no significant differences regarding charge variant profiles neither by clEF nor by CEX-HPLC. The product purity by SE-HPLC is highly similar whatever the F/T number. Fragments content with respect to CGE (reduced and non-reduced) is also comparable. To conclude, the freeze and thaw at −20° C. (up to 3 times) of the UFDF-3 product sample has no significant impact.

### Case Study

### Case Study 1

Inputs: Volume to process=80 L and Concentration=75 g/L

In this particular case, main concerns are the retentate tank capacity, the cassettes surface and hence the related pump speed. The first step is to define the surface required for this UFDF-3 step by targeting 25 L/m² (VLF set point):

$$\frac{Volume to process}{Target VLF} = \frac{80 L}{25 L \text{/} {m}^{2}} = 3.2 {m}^{2}$$

The next calculation will be to assess if by using this surface, the VLF will still fit with the specifications:

$$\frac{Volume to process}{Cassettes surface} = \frac{80 L}{2.85 {m}^{2}} = 28 L \text{/} {m}^{2}$$

This VLF remains within specifications (≤30 L/m2) even it is higher than the set point of 25 L/m². The second step is to ensure if the system pump speed is suitable with this cassettes surface taking into account the maximum feed flow rate of 315 LMH (i.e. 290 LMH CFR). Even if the feed flow rate to be applied (898 L/h) is closed to the maximum pump speed limit (≤1000 L/h), it remains suitable for this UFDF-3 step. The third step is to estimate the volume at the start of the diafiltration. Indeed, this volume should be lower than 50 L to fit with the maximum retentate tank capacity (≤55 L). Knowing that the diafiltration concentration set point is 100 g/L, the DF volume is:

$$\frac{{C}_{i n i t i a l e} \times {V}_{i n i t i a l}}{{C}_{D F}} = \frac{75 g \text{/} L \times 80 L}{100 g \text{/} L} = 60 L$$

As explained earlier, if this volume does not fit with the tank capacity (higher than 55 L), the target DF concentration should be 110 g/L instead of 100 g/L set point:

$$\frac{{C}_{i n i t i a l e} \times {V}_{i n i t i a l}}{{C}_{D F}} = \frac{75 g \text{/} L \times 80 L}{110 g \text{/} L} = 54.5 L$$

Moreover, this calculated volume corresponds to the total retentate volume without including the system hold-up volume (HUV) which includes the flow path void volume (FVV) and the cassette/device void volume (DVV)

The DF volume actually contained into the retentate tank is thus:

$${V}_{D F} - System HUV = {V}_{D F} - ( FVV + ( 2 \times {DVV}_{1.14 {m}^{2}} + 1 \times {DVV}_{0.57 {m}^{2}} ) ) = 54.5 L - ( 0.6 \; 50 L + ( 2 \times 0.2 \; 27 L + 1 \times 0.1 \; 18 L ) ) = 54.5 L - 1.222 L = 53.3 L$$

This volume fit with the retentate tank maximum capacity and can be processed (55 L).

The final step is to estimate the preflushed volume into the tank assuming a related concentration of 220 g/L to ensure that it is higher than the minimum retentate tank capacity:

$$\frac{{C}_{i n i t i a l e} \times {V}_{i n i t i a l}}{{C}_{preflushed}} = \frac{75 g \text{/} L \times 80 L}{220 g \text{/} L} = 27.3 L$$

There is no issue regarding the minimum retentate tank capacity. It is not a concern with such volumes.

### Case Study 2

Inputs: Volume to process=45 L, Concentration=65 g/L

In this case study, there is only one main concern which is the retentate tank capacity and more precisely its minimum recirculation volume. The same exercise as the case study 1 above is performed to assess the suitability of these inputs by considering same parameters.

The first step is to define the surface required for this UFDF-3 step by targeting 25 L/m2 (VLF set point):

$$\frac{Volume to process}{Target VLF} = \frac{45 L}{25 L \text{/} {m}^{2}} = 1.8 {m}^{2}$$

Based on two cassettes sizes suitable with the manufacturing scale (0.57 m2 and 1.14 m2) and in order to achieve this required surface, 2×1.14 m2 is used (total surface of 2.28 m2). The next calculation will be to assess if by using this surface, the VLF will still fit with the specifications:

$$\frac{Volume to process}{Cassettes surface} = \frac{45 L}{2.28 L \text{/} {m}^{2}} = 20 {m}^{2}$$

This VLF remains within specifications (≤30 L/m2) even it is lower than the set point of 25 L/m2. In this case, the feed flow rate to applied targeting a set point of 315 LMH (i.e. 290 LMH CFR) is not on the critical path. Indeed the maximum pump capacity (≤1000 L/h) is not reached using this surface (2.28 m2). It is nevertheless calculated for information only:

Feed flow rate×Cassettes surface=315 LMH×2.28 m²=718 L/h

Within the same way, the diafiltration step can be performed at 100 g/L without any issue. Anyway, the volume content into the retentate tank at this stage does not reach the minimum tank capacity. However, the critical point to consider here is the pre-flushed volume remaining into the retentate tank at the end of concentration. The final step is to estimate this volume assuming a pre-flushed concentration of 220 g/L. The goal is to ensure that it is higher than the minimum retentate tank capacity:

$$\frac{{C}_{i n i t i a l e} \times {V}_{i n i t i a l}}{{C}_{preflushed}} = \frac{65 g \text{/} L \times 45 L}{220 g \text{/} L} = 13.3 L$$

This calculated volume corresponds to the total retentate volume without including the HUV thus the pre-flushed volume actually contained into the retentate tank is:

$${V}_{pre - flushed} - System HUV = {V}_{pre - flushed} - ( FVV + ( 2 \times {DVV}_{1.14 {m}^{2}} ) ) = 13.3 L - ( 0.6 \; 50 L + ( 2 \times 0.2 \; 27 L ) ) = 13.3 L - 1.104 L = 12.2 L$$

Although it is really closed to the minimum, this pre-flushed volume fit with the retentate tank minimum capacity and can be processed.

To conclude, the Table 11 is a summary of process parameters to be applied for performing this UFDF-3 case study:

TABLE 11
summary of UFDF-3 case studies

| | Parameters | Operating ranges | | Case study 1 | | Case Study 2 | |
|---|---|---|---|---|---|---|---|
| Inputs | Volume | ≤80 | L | 80 | L | 40 | L |
| | Concentration | ≥65 g/L-≤75 g/L | | 75 | g/L | 65 | g/L |
| Process | Cassette Surface | ≤2 × 1.14 m2 + | | 2.85 m² | | 1.71 m² | |
| parameter | | 1 × 0.57 m² | | (2 × 1.14 m² + | | (1 × 1.14 m² + | |
| to applied | | | | 1 × 0.57 m² | | 1 × 0.57 m²) | |
| | VLF | 25 L/m2 set point | | 28 | L/m² | 23 | L/m² |
| | | ≤30 L/m2 | | | | | |
| | CFR | 290 LMH set point | | 290 LMH | | | |
| | | (≥240 LMH-≤360 | | | | | |
| | | LMH) | | | | | |
| | Feed flow rate | 315 LMH set point | | 315 LMH | | | |
| | | (≥260 LMH-≤390 | | | | | |
| | | LMH) | | | | | |
| | Pump speed | ≤1000 | L/h | 898 | L/h | 539 | L/h |

| | | | | | |
|---|---|---|---|---|---|
| DF concentration | 100 g/L set point | 110 | g/L | 100 | L/h |
| | (≤110 g/L) | | | | |

### UFDF-3 for High Concentration of and Antibody Solution—5000 L Processing

### Materials, Equipment and Methods

Buffers were prepared at room temperature (≥+19 and ≤+25° C.) and pH was adjusted to the target at room temperature, the list of used buffer for the concentration of Ab1 with the developed UFDF-3 starting from the an antibody solution with an Ab concentration between about 65 and about 75 g/L are listed in Table 12.

TABLE 12
List of buffers used for the UFDF-3 and formulation process

| Buffer description | Step |
|---|---|
| 5 mM L-Histidine pH 6.0 | Equilibration |
| 25 mM L-Histidine, 150 mM | Diafiltration, |
| L-Arginine-HCl, pH 6.0 | flush |
| 25 mM L-histidine, 150 mM | Excipient addition |
| L-arginine-HCl, 2.5% v/v | |
| Polysorbate 80, pH 6.0 | |

TABLE 13
Characteristics of equilibration buffer

| Parameter | Set Point | Acceptable Range | Unit |
|---|---|---|---|
| pH (20-25° C.) | 6.0 | ≥5.9-≤6.1 | NA |
| Conductivity (24-26° C.) | 0.30 | ≥0.15-≤0.45 | mS/cm |
| Bioburden | — | ≤3 (Alert Limit) | CFU/10 mL |
| | | ≤50 (Action Limit) | |
| Endotoxin | — | ≤1 | EU/mL |

TABLE 14
Characteristics of diafiltration buffer

| Parameter | Set Point | Acceptable Range | | Unit |
|---|---|---|---|---|
| pH (20-25° C.) | 6.0 | ≥5.9-≤6.1 | | NA |
| Conductivity (24-26° C.) | 12.2 | ≥12.0-≤12.4 | | mS/cm |
| Osmolality¹ | 293 | NA | | mOsm/kg |
| Bioburden | — | ≤3 | (Alert Limit) | CFU/10 mL |
| | | ≤50 | (Action Limit) | |

| | | | |
|---|---|---|---|
| Endotoxin | — | ≤1 | EU/mL |

TABLE 15
Characteristics of formulation buffer

| Parameter | Set Point | Acceptable Range | | Unit |
|---|---|---|---|---|
| pH (20-25° C.) | 6.0 | ≥5.9-≤6.1 | | NA |
| Conductivity (24-26° C.) | 11.5 | ≥11.1-≤11.9 | | mS/cm |
| Osmolality | 313 | NA | | mOsm/kg |
| Bioburden | — | ≤3 | (Alert Limit) | CFU/10 mL |
| | | ≤50 | (Action Limit) | |

| | | | |
|---|---|---|---|
| Endotoxin | — | ≤1 | EU/mL |

The prepared buffers were tested for bioburden and then filtered through 0.22 μm filter and tested for Endotoxins post filtration.

The TFF cassettes with nominal molecular weight limit (NMWL) of 30 KDa were used for the UFDF-3 at 5000 L scale antibody production (see paragraph “Downstream process (DSP)” for steps details). The surface required can be recalculated according to the material quantity at the beginning of the step. Single-use or reusable cassettes can be used.

In our case the starting material for the developed UFDF-3 step was the antibody solution obtained by a after the UFDF2 of the Downstream process (DSP) described before, which had an antibody concentration between about 65 and about 75 g/L.

### UFDF-3 Step Description

The objective of this step was to reach a high concentration of the antibody Ab1 (170 g/L) and to buffer exchange the product into the diafiltration buffer made of 25 mM L-Histidine, 150 mM L-Arginine-HCl, pH6.0) required before excipient addition.

The cassettes need to be installed in the holder following the supplier recommendations (torque value). The number of cassettes to be used depends on the volume of material to be used. This needs to be calculated using the mass balance equations:

$$• Total Product from UFDF 2 ( g ) = UFDF 2 Pool A 280 Concentration ( g \text{/} L ) \times UFDF \text{-} 2 Pool Volume ( L )$$

$$• Total Membrane surface required ( {m}^{2} ) = \frac{Total Product from VFUFDF \text{-} 2 ( g )}{Maximum Loading factor ( g \text{/} {m}^{2} )}$$

$$• Number of 0.57 {m}^{2} cassettes required ( rounded up value ) = \frac{Total Membrane surface required ( {m}^{2} )}{Membrane surface ( {m}^{2} ) = 0.57}$$

The membrane surface available are 0.57 m² and 1.14 m². Regarding the 5000 L scale production, the recovery and expected volume UFDF-3 load to be processed can be from 40 L to 80 L. Prior to any UFDF-3 run, a sanitization needs to be performed if the system flow path and/or cassettes are not single-use or non-gamma irradiated. After the sanitization, the cassettes was rinsed using water for injection (WFI) and then equilibrated using the 5 mM L-Histidine pH 6.0 buffer (≥10 L/m²) until buffer pH and conductivity of retentate and permeate lines are within specifications of the equilibration buffer (Table 13). After the loading of Ab1 in the retentate container, the first ultrafiltration (UF1) operation can directly start. The product needs to be concentrated from about 70 g/L to approximatively 100 g/L.

The step of first concentration is followed by the diafiltration (DF), the product needs to be buffer exchanged to the diafiltration buffer 25 mM L-Histidine, 150 mM L-Arginine-HCl pH 6.0 using at minimum 6 DVs. The pH and conductivity of permeate need to be checked at this point and need to be in the DF buffer specifications (Table 14) before to proceed to next step. After the ultrafiltration 2 (UF2) operation is performed, the product must reach the exact concentration calculated before to start UFDF-3 based on the quantity of product in UFDF-3 load and the volume required for the flush. The pre-flushed product needs to be sufficiently concentrated to allow an effective flush of the lines and the cassettes and hence to reach the concentration of 170 g/L at the end.

As described in paragraph “UFDF-3 for high concentration of and antibody solution—condition selection”, a concentration of 260 g/L was reached with a feed pressure <3 bars without any impact on product quality. Due to the high viscosity of the product, a gradient with 2 levels of viscosity was observed which induce an important feed pressure increase. During UF2 of the UFDF-3 stage the pressure and viscosity increased with the increase of the product concentration. Therefore the feed pump flow rate was decreased to maintain the pressure approximately at 0.8 bars. When the minimum pump flow is reached, the retentate valve is opened to maintain the retentate pressure >0.0 bar and allow the TMP to increase to approximately 1.5 bars and a feed pressure up to 3 bars, to be able to concentrate the product to the target. At the end of ultrafiltration 2, only the feed pressure will be controlled to avoid to exceed the maximum.

Due to the high concentration of the product before flush (approximatively 220 g/L), the 0.2 μm filtration was performed just after the pool (product after UF2 and flush), in order to maintain a low bioburden level on UFDF-3 product (at 170 g/L), followed immediately by the excipient addition and concentration adjustment.

The flushing volume for this stage was 3 HUV. The flush volume must be re-calculated after ultrafiltration 2 based on the final concentration reached, to be optimal to reach the defined UFDF-3 concentration target after flush while maintaining an acceptable product recovery (Step yield specification 85%).

### CONCLUSION

In summary the developed UFDF3 steps allowed to concentrate an antibody solution with a starting antibody concentration of about 70 g/L up to about 170 g/L. The steps of the developed UFDF3 are summarized below:

### Sanitation:

Pre-Use water for injection (WFI) flush
Sanitation with 0.5 NaOH-30 min recirculation
Pre-use WFI rinsing 0.1 mS/cm
Feed pressure: ≤3 bars
TMP target 0.8 bars (≥0.6 bars-≤1. bars)

### Equilibration:

Equilibration buffer: 5 mM L-Histidine pH 6.0
Feed pressure: ≤3 bars
TMP target 0.8 bars (≥0.6 bars-≤1. bars)

### Loading:

Volumetric loading factor: target 25 L/m2 (≤30 L/m2)
Concentration: ≥65 g/L-≤75 g/L

### First Ultrafiltration:

Cross flow rate: target 290 LMH (≥100 LMH-≤325 LMH)
Feed flow rate: target 315 LMH (≥110 LMH-≤350 LMH)
Feed pressure: ≤3 bars
TMP: target 0.8 bars (≥0.6 bars-≤1.0 bars)
Concentration: target 100 g/L (≥90 g/L-≤110 g/L)

### Diafiltration:

Cross flow rate: target 290 LMH (≥100 LMH-≤325 LMH)
Feed flow rate: target 315 LMH (≥110 LMH-≤350 LMH)
Feed pressure: ≤3 bars
TMP: target 0.8 bars (≥0.6 bars-≤1.0 bars)
Diafiltration buffer: 25 mM L-Histidine, 150 mM L-Arginine-HCl pH 6.0, performed at ≥6 DVs
   Second ultrafiltration:
Cross flow rate: target 290 LMH (≥7 LMH≤325 LMH)
Feed flow rate: target 315 LMH (≥7 LMH-≤350 LMH)
Feed pressure: 3 bars
TMP: ≤1.5 bars
Concentration: ≤260 g/L

### Flushing:

Flushing buffer: 25 mM L-Histidine, 150 mM L-Arginine-HCl pH 6.0-≥3 HUV
Concentration reached: ≥162 g/L-≤179 g/L

The UFDF-3 product at 170 g/L was then formulated with Polysorbate 80 and diluted to 150 g/L. In particular the final antibody formulation contained 150 g/L of Ab1, 25 mM of L-histidine-HCl, 150 mM of L-arginine-HCl and Polysorbate 80 present at a concentration of about 0.036% (w/v).

1. A process for obtaining a highly concentrated antibody solution comprising the steps of subjecting a clarified cell harvest to an affinity chromatography step, and subjecting the obtained eluate to at least two ion exchange chromatography steps and at least three UF/DF steps.

2. The process of claim 1, wherein said highly concentrated antibody solution has an antibody concentration equal to or greater than about 120 g/L.

3. The process of claims 1 and 2, wherein said at least three UF/DF steps are performed with a tangential flow filtration cassette and comprise a first UF/DF performed after the first of said at least two ion exchange chromatography, a second UF/DF performed after the second of said at least two ion exchange chromatography, and a third UF/DF performed after the second UF/DF, and wherein said highly concentrated antibody solution has an antibody concentration equal to or greater than about 150 g/L.

4. The process of claim 3, wherein said third UF/DF comprises the steps of
(a) equilibration of the cassette by an equilibration buffer;
(b) loading of the cassette with an antibody solution with antibody concentration comprised between about 50 g/L and about 90 g/L;
(c) first ultrafiltration to concentrate the antibody to a concentration comprised between about 80 g/L and about 120 g/L;
(d) diafiltration using a diafiltration buffer;
(e) second ultrafiltration to concentrate the antibody to a concentration comprised between about 200 g/L and about 300 g/L;
(f) flushing of the cassette with a flushing buffer;
(g) obtaining a highly concentrated antibody solution with antibody concentration comprised between about 150 g/L and about 200 g/L.

5. The process of claim 4, wherein the antibody solution loaded onto the third UFDF cassette has an antibody concentration of about 70 g/L and/or the first ultrafiltration concentrates the antibody to a concentration of about 100 g/L and/or the second ultrafiltration concentrates the antibody to a concentration of about 260 mg/mL and/or the obtained highly concentrated antibody solution has an antibody concentration of about 170 g/L.

6. The process of any one of the preceding claims, wherein the third UF/DF is performed using an equilibration buffer comprising histidine-HCl at a concentration of about 5 mM and having pH about 6, a diafiltration buffer comprising histidine-HCl at a concentration of about 25 mM and arginine-HCl at a concentration of about 150 mM and having pH of about 6 and flushing buffer comprising histidine-HCl at a concentration of about 25 mM and arginine-HCl at a concentration of about 150 mM and having pH of about 6.

7. The process of any one of the preceding claims wherein said affinity chromatography is protein A affinity chromatography.

8. The process of any one of the proceeding claims, wherein said two steps of ion exchange chromatography steps comprise a first step of cation exchange chromatography and a second step of anion exchange chromatography.

9. A stable pharmaceutical formulation obtained by adding excipients to said highly concentrated antibody solution obtained by the process of any one of claims 1 to 8.

10. The stable pharmaceutical formulation of claim 9, comprising an a antibody or fragment thereof present within said pharmaceutical formulation at a concentration of about 150 g/L, histidine-HCl buffer present within said pharmaceutical formulation at a concentration of about 25 mM, arginine-HCl present within said pharmaceutical formulation at a concentration of about 150 mM and Polysorbate 80 present within said pharmaceutical formulation at a concentration of about 0.036% (w/v).

11. A process of production of a bulk drug substance or a drug product comprising the steps of:
(a) Protein A chromatography of a clarified cell harvest comprising an antibody;
(b) Viral inactivation of the resulting protein A eluate;
(c) Neutralization of the protein A eluate to pH 5.2, followed by 0.2 μm filtration;
(d) Cation exchange chromatography of the neutralized protein A eluate, followed by 0.2 μm filtration;
(e) First UF/DF of the cation exchange chromatography eluate, followed by 0.2 μm filtration;
(f) Anion exchange chromatography in flow through mode performed by membrane adsorption, followed by 0.2 am filtration;
(g) Viral nanofiltration;
(h) Second UF/DF of the nanofiltrated solution, followed by 0.2 um filtration;
(i) Third UF/DF of the antibody solution obtained by the second UF/DF according to the processes of claims 1 to 6, followed by 0.2 um filtration;
(j) Obtaining a stable pharmaceutical formulation by adding excipients to the highly concentrated antibody solution obtained by the third UF/DF, followed by 0.2 um filtration.

12. The process of claim 11, wherein said third UF/DF is performed according to claim 6, and said stable pharmaceutical formulation is the formulation of claim 9 or 10.
